(19) United States
(12) Patent Application Publication
(10) Pub. No.: US 2025/0304632 A1
(43) Pub. Date: Oct. 2, 2025

(54) MUTANT STRAIN OF TRICHODERMA REESEI AND METHOD OF PRODUCING PROTEIN USING SAME

(71) Applicant: Toray Industries, Inc., Tokyo (JP)

(72) Inventors: Yusuke Kagawa, Kamakura-shi Kanagawa (JP); Haruka Saito, Kamakura-shi Kanagawa (JP); Shingo Hiramatsu, Kamakura-shi Kanagawa (JP); Katsushige Yamada, Kamakura-shi Kanagawa (JP)

(73) Assignee: Toray Industries, Inc., Tokyo (JP)

(21) Appl. No.: 19/235,116

(22) Filed: Jun. 11, 2025

Related U.S. Application Data
(62) Division of application No. 17/271,940, filed on Feb. 26, 2021, now Pat. No. 12,358,958.

(30) Foreign Application Priority Data
Aug. 29, 2018 (JP) 2018-160156

(51) Int. Cl. C07K 14/37; C12N 1/14; C12N 9/42; C12P 19/00; C12R 1/885

(52) U.S. Cl. CPC C07K 14/37; C12N 1/14; C12N 9/2437; C12P 19/00; C12N 2500/34; C12R 2001/885

(57) ABSTRACT

A mutant strain of Trichoderma reesei, the mutant strain having a mutation that eliminates or reduces a function of a polypeptide consisting of the amino acid sequence represented by SEQ ID No: 6. A method produces a cellulase, the method including a step of cultivating the mutant strain of Trichoderma reesei, the mutant strain having a mutation that eliminates or reduces a function of a polypeptide consisting of the amino acid sequence represented by SEQ ID No: 6.

### CROSS-REFERENCE TO RELATED APPLICATIONS

The application is a divisional application of U.S. application Ser. No. 17/271,940, filed Feb. 26, 2021, which is a 35 U.S.C. § 371 of International Application No. PCT/JP2019/033642, filed Aug. 28, 2019, which claims priority to Japanese Patent Application No. 2018-160156, filed Aug. 29, 2018, each of which is incorporated herein by reference in its entirety.

### SEQUENCE LISTING

The instant application contains a Sequence Listing which has been submitted electronically and is hereby incorporated by reference in its entirety. Said electronic copy, created on Jun. 10, 2025, is named 20210226 NBC-21-1095SequenceListing.xml and is 66,866 bytes in size.

### TECHNICAL FIELD

This disclosure relates to a Trichoderma reesei mutant strain having an enhanced protein-producing ability and to a method of protein production using the mutant strain.

### BACKGROUND

Trichoderma reesei is known to have a high protein-producing ability, and studies have heretofore been made on protein production using Trichoderma reesei. Trichoderma reesei is especially excellent in terms of the ability to produce a cellulase, which is classified as a saccharifying enzyme, among proteins. For example, to further enhance cellulase production amount, investigations such as overexpression or deletion of a factor that controls cellulase production and investigations on cultivation conditions for cellulase production are being conducted.

Juliano P, Single nucleotide polymorphism analysis of a Trichoderma reesei hyper-cellulolytic mutant developed in Japan, Bioscience, Biotechnology, and Biochemistry, Volume 77, 2013, Issue 3, P534-543 describes that a mutant strain of Trichoderma reesei which has a high cellulase-producing ability was acquired by reducing the function of Cre1, which is a transcription factor repressing cellulase production, among the cellulase-production-controlling factors of Trichoderma reesei.

Meanwhile, Antonella A, Regulation of cellulase and hemicellulose gene expression in fungi, Current Genomics, Volume 14, 2013, P230-249 describes a method of cultivating Trichoderma reesei in a culture medium to which glucose or lactose has been added, as a method of improving the production amount of a cellulase of Trichoderma reesei.

As described above, a transcription factor which is one of protein-production-controlling factors in Trichoderma reesei has been identified, but this is considered to be merely a part of the control mechanism. Thus, it could be helpful to acquire a mutant strain of Trichoderma reesei having a further enhanced protein-producing ability by making a search for a novel mechanism controlling protein production by Trichoderma reesei, and provide a method of protein production using the mutant strain of Trichoderma reesei.

### SUMMARY

We wondered whether a gene that had been unknown and was capable of bringing about an increase in protein production could be specified, then the amount of proteins to be produced by Trichoderma reesei could be further increased. We thus discovered that an improvement in protein-producing ability can be attained by cultivating a mutant strain of Trichoderma reesei in which the function of one or more polypeptides selected from polypeptides consisting of the amino acid sequences represented by SEQ ID NOs: 4, 5, and 6 has been eliminated or reduced.

We thus provide (1) to (15):

(1) A mutant strain of Trichoderma reesei, the mutant strain having a mutation that eliminates or reduces a function of a polypeptide consisting of the amino acid sequence represented by any of SEQ ID NOs: 4 to 6.

(2) The mutant strain according to (1), in which the mutation is a mutation that deletes an HSF-type DNA-binding domain of a polypeptide consisting of the amino acid sequence represented by SEQ ID NO: 4.

(3) The mutant strain according to (2), in which the mutation is a frameshift mutation accompanying a mutation in a region nearer to the N-terminal side than the HSF-type DNA-binding domain.

(4) The mutant strain according to (3), in which the mutation is a frameshift mutation due to a mutation in which a histidine residue at the 30th residue from the N-terminal side in the amino acid sequence represented by SEQ ID NO: 4 is changed to a residue of an amino acid other than histidine.

(5) The mutant strain according to (1), in which the mutation is a mutation that deletes a TLD domain of a polypeptide consisting of the amino acid sequence represented by SEQ ID NO: 5.

(6) The mutant strain according to (5), in which the mutation is a frameshift mutation accompanying a mutation in a region nearer to the N-terminal side than the TLD domain.

(7) The mutant strain according to (6), in which the mutation is a frameshift mutation due to a mutation in which a glutamine residue at the 3rd residue from the N-terminal side in the amino acid sequence represented by SEQ ID NO: 5 is changed to a residue of an amino acid other than glutamine.

(8) The mutant strain according to (1), in which the mutation is a mutation of an amino acid sequence of an F-box domain region of a polypeptide consisting of the amino acid sequence represented by SEQ ID NO: 6.

(9) The mutant strain according to (8), in which the mutation is a deletion of the polypeptide consisting of the amino acid sequence represented by SEQ ID NO: 6, the deletion being caused by a frameshift mutation accompanying a mutation of the amino acid sequence of the F-box domain region.

(10) The mutant strain according to (9), in which the mutation is a frameshift mutation due to a mutation in which an alanine residue at the 167th residue from the N-terminal side in the amino acid sequence represented by SEQ ID NO: 6 is changed to a residue of an amino acid other than alanine.

(11) A method of producing a protein, the method including a step of cultivating the mutant strain according to any one of (1) to (10).

(12) A method of producing a protein, the method including a step of cultivating the mutant strain according to any one of (1) to (10), in a culture medium at least including lactose.

(13) A method of producing a cellulase, the method including a step of cultivating the mutant strain according to any one of (1) to (10).

(14) A method of producing a cellulase, the method including a step of cultivating the mutant strain according to any one of (1) to (10), in a culture medium at least including lactose.

(15) A method of producing a sugar, the method including:
a step of producing a cellulase by the method of producing a cellulase according to (13) or (14); and
a step of saccharifying a cellulose-containing biomass using the cellulase obtained in the step.

The mutant strain of Trichoderma reesei in which the function of a polypeptide consisting of the amino acid sequence represented by any of SEQ ID NOs: 4 to 6 has been eliminated or reduced has an improved protein-producing ability and is capable of highly efficiently producing a protein compared to the parent strain into which the mutation has not been introduced. Furthermore, when the produced proteins are cellulases, an unexpected effect that the cellulases have improved various specific activities is also obtained.

### DETAILED DESCRIPTION

Our strains and methods are characterized in that a mutation is introduced into a parent strain of Trichoderma reesei, which is a microorganism originally having an excellent protein-producing ability, to thereby further enhance the protein-producing ability. Specifically, we provide a mutant strain of Trichoderma reesei, the mutant strain being characterized by having a mutation which eliminates or reduces the function of a polypeptide consisting of the amino acid sequence represented by any of SEQ ID NOs: 4 to 6.

The parent strain of Trichoderma reesei is not limited to wild strains, and mutant strains that have been improved to have an increased protein-producing ability can also be favorably used as the parent strain. For example, a mutant strain having an improved protein production property obtained by performing a mutation treatment with a mutagen, UV irradiation or the like can be utilized as the parent strain. Specific examples of mutant strains usable as the parent strain include the following known mutant strains belonging to Trichoderma reesei: QM6a strain (NBRC31326), QM9123 strain (ATCC24449), QM9414 strain (NBRC31329), PC-3-7 strain (ATCC66589), QM9123 strain (NBRC31327), RutC-30 strain (ATCC56765), CL-847 strain (Enzyme. Microbiol. Technol., 10, 341-346 (1988)), MCG77 strain (Biotechnol. Bioeng. Symp., 8, 89 (1978)), and MCG80 strain (Biotechnol. Bioeng., 12, 451-459 (1982)). QM6a strain, QM9414 strain, and QM9123 strain are available from NBRC (NITE Biological Resource Center), and PC-3-7 strain and RutC-30 strain are available from ATCC (American Type Culture Collection).

The polypeptide consisting of the amino acid sequence represented by SEQ ID NO: 4 is a polypeptide possessed by Trichoderma reesei, and in National Center for Biotechnology Information, this polypeptide has been registered as a predicted protein, partial (EGR45828) that Trichoderma reesei QM6a strain has. The polypeptide consisting of the amino acid sequence represented by SEQ ID NO: 4 is a polypeptide whose function is unknown, but Conserved Domain Architecture Retrieval Tool of National Center for Biotechnology Information discloses that the 86th to 186th amino acid residues from the N-terminal side are a heat shock factor (HSF)-type DNA-binding domain. The HSF-type DNA-binding domain is known to have the function of binding to an upstream region of a gene encoding an HSF, which is a transcription factor controlling the expression of heat shock proteins (Cell, 65(3), 363-366 (1991)). Specific examples of genes encoding the polypeptide consisting of the amino acid sequence represented by SEQ ID NO: 4 include the base sequence represented by SEQ ID NO: 1.

Examples of methods of eliminating or reducing the function of the polypeptide consisting of the amino acid sequence represented by SEQ ID NO: 4 include a method of introducing a mutation that causes a total deletion of an HSF-type DNA-binding domain, a partial deletion of an HSF-type DNA-binding domain, or a total deletion of the polypeptide consisting of the amino acid sequence represented by SEQ ID NO: 4. Specific examples thereof include a method in which a frameshift mutation or a stop codon mutation is introduced into a gene sequence encoding the polypeptide consisting of the amino acid sequence represented by SEQ ID NO: 4, by a deletion, insertion, substitution or the like of a base.

The phrase “deletion of an HSF-type DNA-binding domain” means a total or partial loss of the domain, a change of the whole or some of the domain into different amino acid(s), or a combination of these. More specifically, that phrase means that the amino acid sequence represented by SEQ ID NO: 4 comes to have a sequence identity of 80% or less with respect to the amino acid sequence of the HSF-type DNA-binding domain. The sequence identity thereto is preferably 50% or less, more preferably 20% or less, more preferably 10% or less, more preferably 5% or less, more preferably 3% or less, more preferably 1% or less, and most preferably 0%.

Specific examples of when the function of a polypeptide consisting of the amino acid sequence represented by SEQ ID NO: 4 is eliminated or reduced by a mutation such as deletion, substitution, or addition, that has occurred in an amino acid sequence located in the HSF-type DNA-binding domain include a mutation in the amino acid sequence represented by SEQ ID NO: 4 which results in a partial or total loss of the region ranging from the 86th to 186th residues from the N-terminal side, which corresponds to the HSF-type DNA-binding domain. Specific examples of such mutations include a mutation in the base sequence represented by SEQ ID NO: 1 which causes a frameshift including insertion of one base residue of guanine into the 85th position. This mutation changes the 30th amino acid residue from the N-terminal side in the amino acid sequence represented by SEQ ID NO: 4 from histidine to threonine. The succeeding frameshift causes the translation to end at the 90th amino acid residue from the N-terminal side and results in loss of the amino acid sequence constituting the HSF-type DNA-binding domain.

The polypeptide consisting of the amino acid sequence represented by SEQ ID NO: 5 is a polypeptide possessed by Trichoderma reesei, and in National Center for Biotechnology Information, this polypeptide has been registered as a predicted protein (EGR47155) that Trichoderma reesei QM6a strain has. The polypeptide consisting of the amino acid sequence represented by SEQ ID NO: 5 is a polypeptide whose function is unknown, but Conserved Domain Architecture Retrieval Tool of National Center for Biotechnology Information discloses that the 362nd to 553rd amino acid residues from the N-terminal side are a TLD domain. The function of the TLD domain is unknown. Specific examples of genes encoding the polypeptide consisting of the amino acid sequence represented by SEQ ID NO: 5 include the base sequence represented by SEQ ID NO: 2.

Examples of methods of reducing the function of the polypeptide consisting of the amino acid sequence represented by SEQ ID NO: 5 include a method of introducing a mutation that causes a total deletion of a TLD domain, a partial deletion of a TLD domain, or a total deletion of the polypeptide consisting of the amino acid sequence represented by SEQ ID NO: 5. Specific examples thereof include a method in which a frameshift mutation or a stop codon mutation is introduced into a gene sequence encoding the polypeptide consisting of the amino acid sequence represented by SEQ ID NO: 5, by a deletion, insertion, substitution or the like of a base.

The phrase “deletion of a TLD domain” means a total or partial loss of the domain, a change of the whole or some of the domain into different amino acid(s), or a combination of these. More specifically, that phrase means that the amino acid sequence represented by SEQ ID NO: 5 comes to have a sequence identity of 80% or less with respect to the amino acid sequence of the TLD domain. The sequence identity thereto is preferably 50% or less, more preferably 20% or less, more preferably 10% or less, more preferably 5% or less, more preferably 3% or less, more preferably 1% or less, and most preferably 0%.

Specific examples of when the function of a polypeptide consisting of the amino acid sequence represented by SEQ ID NO: 5 is eliminated by a mutation such as deletion, substitution, or addition, that has occurred in an amino acid sequence located in the TLD domain include a mutation in the amino acid sequence represented by SEQ ID NO: 5 which results in a partial or total loss of the region ranging from the 362nd to 553rd residues from the N-terminal side, which corresponds to the TLD domain. Specific examples of such mutations include a frameshift mutation in the base sequence represented by SEQ ID NO: 2 which causes insertion of the 46 base residues represented by SEQ ID NO: 27 into the 6th position. This mutation changes the glutamine residue at the 3rd residue from the N-terminal side in the amino acid sequence represented by SEQ ID NO: 5 into arginine. The translation ends at this position, and the amino acid sequence constituting the TLD domain is lost.

The polypeptide consisting of the amino acid sequence represented by SEQ ID NO: 6 is a polypeptide possessed by Trichoderma reesei, and in National Center for Biotechnology Information, this polypeptide has been registered as a predicted protein (EGR48056) that Trichoderma reesei QM6a strain has. The polypeptide consisting of the amino acid sequence represented by SEQ ID NO: 6 is a polypeptide whose function is unknown, but Conserved Domain Architecture Retrieval Tool of National Center for Biotechnology Information discloses that the 130th to 172nd amino acid residues from the N-terminal side are an F-box domain. The F-box domain is known to be a domain present in proteins which control the cell cycle (Proc. Natl. Acad. Sci., 95, 2417-2422 (1998)). Specific examples of genes encoding the polypeptide consisting of the amino acid sequence represented by SEQ ID NO: 6 include the base sequence represented by SEQ ID NO: 3.

Examples of methods of reducing the function of the polypeptide consisting of the amino acid sequence represented by SEQ ID NO: 6 include a method of introducing a mutation that causes a total deletion of an F-box domain, a partial deletion of an F-box domain, or a total deletion of the polypeptide consisting of the amino acid sequence represented by SEQ ID NO: 6. Specific examples thereof include a method in which a frameshift mutation or a stop codon mutation is introduced into a gene sequence encoding the polypeptide consisting of the amino acid sequence represented by SEQ ID NO: 6, by a deletion, insertion, substitution or the like of a base.

The phrase “deletion of an F-box domain” means a total or partial loss of the domain, a change of the whole or some of the domain into different amino acid(s), or a combination of these. More specifically, that phrase means that the amino acid sequence represented by SEQ ID NO: 6 comes to have a sequence identity of 80% or less with respect to the amino acid sequence of the F-box domain. The sequence identity thereto is preferably 50% or less, more preferably 20% or less, more preferably 10% or less, more preferably 5% or less, more preferably 3% or less, more preferably 1% or less, and most preferably 0%.

Specific examples of when the function of a polypeptide consisting of the amino acid sequence represented by SEQ ID NO: 6 is eliminated by a mutation such as deletion, substitution, or addition, that has occurred in an amino acid sequence located in the F-box domain include a frameshift mutation in the base sequence represented by SEQ ID NO: 3 which results in a loss of one cytosine base residue which is the 499th residue. This mutation changes the 167th amino acid residue from the N-terminal side in the amino acid sequence represented by SEQ ID NO: 6 from alanine to arginine. The succeeding frameshift causes the translation to end at the 193rd residue from the N-terminal side, resulting in loss of the amino acid sequence constituting the F-box domain.

Another method that may be used to reduce the function of the polypeptide consisting of the amino acid sequence represented by any of SEQ ID NOs: 4 to 6 is to introduce a mutation which diminishes or inhibits the expression of the polypeptide. Specifically, the mutation that diminishes or inhibits the expression of the polypeptide may be one introduced into the promoter or terminator region of a gene encoding the amino acid sequence represented by any of SEQ ID NOs: 4 to 6. In general, the promoter and terminator regions correspond to a region of hundreds of bases in length before and after the gene participating in transcription.

To introduce such mutations into the gene, use can be made of existing genetic mutation methods such as a mutation treatment with a known mutagen or with UV irradiation or the like, gene recombination such as homologous recombination using a selection marker, and a mutation by a transposon.

Our mutant strain is only required to satisfy that the function of at least one or more polypeptides among polypeptides consisting of the amino acid sequences of SEQ ID NOs: 4 to 6 has been eliminated or reduced. The function of two or all of these polypeptides may have been eliminated or reduced. There are no particular limitations on combinations of polypeptides whose functions have been eliminated or reduced, and any of the following mutant strains are included in the mutant strain: a mutant strain of Trichoderma reesei in which the functions of the polypeptides consisting of the amino acid sequences represented by SEQ ID NOs: 4 and 5 have been eliminated or reduced; a mutant strain of Trichoderma reesei in which the functions of the polypeptides consisting of the amino acid sequences represented by SEQ ID NOs: 4 and 6 have been eliminated or reduced; and a mutant strain of Trichoderma reesei in which the functions of the polypeptides consisting of the amino acid sequences represented by SEQ ID NOs: 5 and 6 have been eliminated or reduced.

The mutant strain may be a mutant strain in which the functions of all the three polypeptides consisting of the amino acid sequences represented by SEQ ID NOs: 4 to 6 have been eliminated or reduced. The mutant strain in which the functions of all the three polypeptides consisting of the amino acid sequences represented by SEQ ID NOs: 4 to 6 have been eliminated or reduced can be acquired by subjecting spores of Trichoderma reesei as a parent strain to a genetic mutation treatment with nitrosoguanidine (NTG), ethylmethanesulfonic acid (EMS), UV and the like, and analyzing the genes of the resultant mutant strains to collect a mutant strain having the mutation by screening.

Since the mutant strain has an enhanced protein-producing ability compared to the parent strain into which the mutation has not been introduced, a culture solution of the mutant strain has a higher protein concentration than a culture solution obtained by cultivating the parent strain not having the mutation under the same cultivation conditions. When the protein is an enzyme, the enzyme has enhanced specific activity. The increasing rate in protein concentration and the increasing rate in enzyme specific activity are not particularly limited so long as the concentration and the specific activity have increased. It is, however, preferable that the increasing rates are 20% or larger.

Besides having a mutation that eliminates or reduces the function of the polypeptide consisting of the amino acid sequence represented by any of SEQ ID NOs: 4 to 6, our mutant strain may have a mutation which improves protein production amount and/or lowers the viscosity of culture solutions to inhibit the degree of saturation of oxygen dissolved in the culture solutions from decreasing. Specific examples thereof include a genetic mutation which reduces the function of the polypeptide represented by any of SEQ ID NOs: 7, 9, and 11.

The polypeptide consisting of the amino acid sequence represented by SEQ ID NO: 7 is a polypeptide possessed by Trichoderma reesei and has been registered at National Center for Biotechnology Information as predicted protein EGR50654 possessed by Trichoderma reesei QM6a strain. The polypeptide consisting of the amino acid sequence represented by SEQ ID NO: 7 is a polypeptide whose function is unknown, but Conserved Domain Architecture Retrieval Tool of National Center for Biotechnology Information discloses that the 95th to 277th amino acid residues from the N-terminal side have Middle domain of eukaryotic initiation factor 4G domain (hereinafter referred to as MIF4G domain) and the 380th to 485th amino acid residues from the N-terminal side have MA-3 domain. The two domains, MIF4G and MA-3, are known to have the function of binding to DNAs or RNAs (Biochem., 44, 12265-12272 (2005); Mol. Cell. Biol., 1, 147-156 (2007)). It is presumed from those disclosures that the polypeptide consisting of the amino acid sequence represented by SEQ ID NO: 7 at least has the function of binding to a DNA and/or an RNA.

Specific examples of genes encoding the polypeptide consisting of the amino acid sequence represented by SEQ ID NO: 7 include the base sequence represented by SEQ ID NO: 8. Examples of genetic mutations which reduce the function of EGR50654 include a total deletion of the MIF4G domain and/or MA-3 domain possessed by EGR50654, a partial deletion of the MIF4G domain and/or MA-3 domain, and a genetic mutation which changes the configuration relationship between the MIF4G domain and the MA-3 domain. Furthermore, the function of the polypeptide consisting of the amino acid sequence represented by SEQ ID NO: 7 can be reduced also by introducing a mutation which diminishes or inhibits the expression of the polypeptide. Specific examples of the deletion of the function of the polypeptide consisting of the amino acid sequence represented by SEQ ID NO: 7 include a mutation in the base sequence represented by SEQ ID NO: 8 which deletes any of the 1,039th to 1,044th bases.

The polypeptide consisting of the amino acid sequence represented by SEQ ID NO: 9 is a polypeptide possessed by Trichoderma reesei and has been registered at National Center for Biotechnology Information as predicted protein EGR44419 possessed by Trichoderma reesei QM6a strain. The polypeptide consisting of the amino acid sequence represented by SEQ ID NO: 9 is a polypeptide whose function is unknown, but Conserved Domain Architecture Retrieval Tool of National Center for Biotechnology Information discloses that the 26th to 499th amino acid residues from the N-terminal side have a Sugar (and other) Transporter domain. It is presumed from this disclosure that the polypeptide consisting of the amino acid sequence represented by SEQ ID NO: 9 at least participates in transport of sugar between the inside and the outside of the fungus bodies.

Specific examples of genes encoding the polypeptide consisting of the amino acid sequence represented by SEQ ID NO: 9 include the base sequence represented by SEQ ID NO: 10. Examples of genetic mutations which reduce the function of EGR44419 include a total deletion of the Sugar (and other) Transporter domain possessed by EGR44419, a partial deletion of the Sugar (and other) Transporter domain, and a genetic mutation which changes the configuration relationship of the Sugar (and other) Transporter domain. Furthermore, the function of the polypeptide consisting of the amino acid sequence represented by SEQ ID NO: 9 can be reduced also by introducing a mutation which diminishes or inhibits the expression of the polypeptide. Specific examples of the deletion of the function of the polypeptide consisting of the amino acid sequence represented by SEQ ID NO: 9 include a mutation in the base sequence represented by SEQ ID NO: 10 which inserts 11 bases at the 1,415th position.

The polypeptide consisting of the amino acid sequence represented by SEQ ID NO: 11 is a polypeptide possessed by Trichoderma reesei and has been registered at National Center for Biotechnology Information as EGR48910 of a beta-adaptin large subunit possessed by Trichoderma reesei QM6a strain. The polypeptide consisting of the amino acid sequence represented by SEQ ID NO: 11 is one of the proteins that constitute adaptor proteins that bind to clathrin which is widely conserved in eucaryotes, and constitute vesicles that take part in transport inside and outside the cells and inside and outside the fungus bodies (Proc. Natl. Acad. Sci. USA., 101, 14108-14113 (2004)).

Specific examples of genes encoding the polypeptide consisting of the amino acid sequence represented by SEQ ID NO: 11 include the base sequence represented by SEQ ID NO: 12. Examples of genetic mutations for EGR48910 include a mutation in the base sequence represented by SEQ ID NO: 12 which changes the cytosine at the 1,080th base into adenine.

We further provide a method of protein production including a step of cultivating the mutant strain.

In the method of protein production, the protein-producing ability is further enhanced by cultivating the mutant strain in a culture medium containing an inducer. Examples of inducers improving the producibility of, in particular, cellulases include lactose, glucose, cellulose, cellobiose, and xylan. Preferred of these are lactose and/or glucose. Especially preferred is lactose.

In adding an inducer to the culture medium, the inducer may be added at any timing, for example, at initiation of the cultivation or in the middle of the cultivation. However, especially when lactose or glucose is added as an inducer, it is preferred to add the inducer in the middle of the cultivation because this addition enables the effect of improving the protein-producing ability to continue during the cultivation. The amount of lactose to be added per day per L of the culture solution is preferably about 1 g to 50 g, more preferably about 3-25 g, especially preferably about 6-20 g. The amount of glucose to be added per day per L of the culture solution is preferably about 1-200 g, more preferably about 5-100 g, especially preferably about 20-80 g.

In adding cellulose, cellobiose, or xylan as an inducer, biomass containing any of these may be added as an inducer. Specific examples of such biomass include not only plants such as seed plant, pteridophyte, bryophyte, algae, and water plant, but also waste building materials. The seed plants are classified into gymnosperms and angiosperms, and both can be used favorably. The angiosperms are further classified into monocotyledons and dicotyledons. Specific examples of the monocotyledons include bagasse, switchgrass, napier grass, erianthus, corn stover, corncob, rice straw, and wheat straw, and preferred specific examples of the dicotyledons include beet pulp, eucalyptus, oak, and white birch.

In using such biomass as an inducer, the biomass may be a pretreated to be used. Methods for the pretreatment are not particularly limited, but, for example, known methods such as acid treatment, sulfuric acid treatment, dilute sulfuric acid treatment, alkali treatment, hydrothermal treatment, subcritical treatment, fine grinding treatment, and steaming treatment can be used. Pulp may be used as the biomass containing cellulose or xylan, which has been subjected to such a pretreatment.

The composition of the culture medium to be used in the step of cultivating the mutant strain of Trichoderma reesei is not particularly limited as long as it is a culture medium composition where the Trichoderma reesei can produce a protein, and a known culture medium composition for filamentous fungi of the genus Trichoderma can be employed. As a nitrogen source, use can be made, for example, of polypeptone, bouillon, CSL, or soybean cake. The inducer for protein production may be added to the culture medium.

Methods for the cultivation are not particularly limited. For example, the mutant strain can be cultivated by liquid culture in which a centrifuge tube, flask, jar fermenter, tank, or the like is used or solid culture in which a plate or the like is used. It is preferred to cultivate Trichoderma reesei under aerobic conditions, and especially preferred among those cultivation methods is submerged culture performed in a jar fermenter or a tank while conducting aeration or stirring. The air flow rate is preferably about 0.1-2.0 vvm, more preferably 0.3-1.5 vvm, especially preferably 0.5-1.0 vvm. The cultivation temperature is preferably about 25-35° C., more preferably 25-31° C. The pH conditions during the cultivation are preferably pH 3.0-7.0, more preferably pH 4.0-6.0. As for cultivation time, the cultivation is conducted under conditions capable of protein production, until the protein is accumulated in a recoverable amount. The cultivation period is usually 24-288 hours, preferably 24-240 hours, more preferably 36-240 hours, still more preferably 36-192 hours.

Although the protein to be produced is not particularly limited, proteins to be excreted from the fungus bodies can be efficiently produced. Preferred of these are enzymes. More preferred are saccharifying enzymes such as cellulases, amylases, invertases, chitinases, and pectinases. Still more preferred are cellulases.

Cellulases that can be produced include various hydrolases, which include enzymes having a decomposition activity against xylan, cellulose, and hemicellulose. Specific examples thereof include cellobiohydrolase (EC 3.2.1.91) which produces cellobiose by hydrolyzing cellulose chains, endoglucanase (EC 3.2.1.4) which hydrolyzes cellulose chains from central portions thereof, β-glucosidase (EC 3.2.1.21) which hydrolyzes cellooligosaccharide and cellobiose, xylanase (EC 3.2.1.8) which is characterized by acting on hemicellulose and, in particular, on xylan, and β-xylosidase (EC 3.2.1.37) which hydrolyzes xylooligosaccharide.

Improvement in protein-producing ability or improvement in cellulase specific activity of our Trichoderma reesei mutant strain compared to the parent strain is ascertained by comparing culture solutions obtained by cultivating the mutant strain and the parent strain under the same conditions in protein concentration or in one or more specific activities selected from the group consisting of β-glucosidase specific activity, β-xylosidase specific activity, and cellobiohydrolase specific activity, the protein concentration and the specific activities being determined by the following methods.

The protein concentration is determined in the following manner. Culture solutions of the mutant strain and parent strain are each centrifuged at 15,000×g for 10 minutes to obtain a supernatant. To 250 μL of Quick Start Bradford protein assay (manufactured by Bio-Rad Laboratories, Inc.) is added 5 μL of a diluted cellulase solution. The mixture is allowed to stand still at room temperature for 15 minutes and then examined for absorbance at 595 nm. The concentration of the protein contained in the saccharifying-enzyme solution is calculated on the basis of a calibration curve obtained using bovine serum albumin solutions as reference solutions.

The β-glucosidase specific activity is determined by the following method. First, for the supernatant of the culture solution, 10 μL of an enzyme dilution is added to 90 μL of 50 mM acetate buffer containing 1 mM p-nitrophenyl-β-glucopyranoside (produced by Sigma-Aldrich Japan), and the mixture is allowed to react at 30° C. for 10 minutes. Then, 10 μL of 2 M sodium carbonate is added and mixed well to stop the reaction, and the increase in absorbance at 405 nm is measured. Finally, release of 1 μmol of p-nitrophenol per minute is defined as 1 U of activity to calculate the specific activity.

The β-xylosidase specific activity is determined by the following method. First, for the supernatant of the culture solution, 10 μL of an enzyme dilution is added to 90 μL of 50 mM acetate buffer containing 1 mM p-nitrophenyl-β-xylopyranoside (produced by Sigma-Aldrich Japan), and the mixture is allowed to react at 30° C. for 30 minutes. Then, 10 μL of 2 M sodium carbonate is added and mixed well to stop the reaction, and the increase in absorbance at 405 nm is measured. Finally, release of 1 μmol of p-nitrophenol per minute is defined as 1 U of activity to calculate the specific activity.

The cellobiohydrolase specific activity is determined by the following method. First, for the supernatant of the culture solution, 10 μL of an enzyme dilution is added to 90 μL of 50 mM acetate buffer containing 1 mM p-nitrophenyl-β-lactopyranoside (produced by Sigma-Aldrich Japan), and the mixture is allowed to react at 30° C. for 60 minutes. Then, 10 μL of 2 M sodium carbonate is added and mixed well to stop the reaction, and the increase in absorbance at 405 nm is measured. Finally, release of 1 μmol of p-nitrophenol per minute is defined as 1 U of activity to calculate the specific activity.

Methods of recovering a protein contained in the culture solution where the mutant strain has been cultivated are not particularly limited, but the protein can be recovered by removing the fungus bodies of the mutant strain from the culture solution. Examples of methods of removing the fungus bodies include centrifugation, membrane separation, and filter press.

Furthermore, when the culture solution in which the mutant strain has been cultivated is used as a protein solution without removing the fungus bodies therefrom, the culture solution is preferably treated so that the mutant strain cannot grow therein. Examples of treatment methods of preventing the fungus bodies from growing include heat treatment, chemical treatment, acid/alkali treatment, and UV treatment.

When the protein is an enzyme, the culture solution from which the fungus bodies have been removed or has been treated so that the fungus bodies cannot grow therein, as stated above, can be used directly as an enzyme solution.

When the protein produced by our method is a cellulase, this cellulase can be used to saccharify cellulose-containing biomass to produce a sugar. The cellulase obtained by cultivating the mutant strain is high especially in β-glucosidase specific activity compared to the cellulase obtained by cultivating the parent strain into which the mutation has not been introduced, and can hence efficiently decompose the cellulose-containing biomass to obtain a sugar solution having a high glucose concentration. Thus, a larger quantity of sugar can be obtained.

The cellulose-containing biomass can be either the same biomass as the cellulose-containing biomass mentioned above as an inducer or a pretreated biomass.

Conditions for the saccharification reaction are not particularly limited. The saccharification reaction temperature is preferably 25-60° C., more preferably 30-55° C. The saccharification reaction time is preferably 2-200 hours. The pH in the saccharification reaction is preferably 3.0-7.0, more preferably 4.0-6.0. In cellulases derived from the genus Trichoderma, the best pH for the reaction is 5.0. Furthermore, since the pH changes during the hydrolysis, it is preferred to add a buffer to the reaction solution or to conduct the reaction while keeping the pH constant by using an acid or an alkali.

When the enzyme is separated and recovered from the saccharified solution, use can be made of a method in which the saccharified solution is filtered with an ultrafiltration membrane or the like to recover the enzyme on the non-permeation side. According to need, a step of removing solid matter from the saccharified solution may be conducted before the filtration. The recovered enzyme can again be used for a saccharification reaction.

### EXAMPLES

Our strains and methods are described specifically below by referring to Examples.

### Reference Example 1: Conditions for Protein Concentration Measurement

Protein concentration measuring reagent used: Quick Start Bradford protein assay (produced by Bio-Rad Laboratories, Inc.)

### Measuring Conditions

Measuring temperature: room temperature
Protein concentration measuring reagent: 250 μL
Culture solution of filamentous fungus: 5 μL
Reaction time: 5 min
Absorbance: 595 nm
Standard: BSA

### Reference Example 2: Conditions for Determination of Specific Activity of Cellulases Conditions for Determination of β-Glucosidase Specific Activity

Substrate: p-nitrophenyl-β-glucopyranoside (produced by Sigma-Aldrich Japan)
Reaction solution: 90 μL of 50 mM acetate buffer containing 1 mM p-nitrophenyl-β-glucopyranoside
Enzyme dilution: 10 μL
Reaction temperature: 30° C.
Reaction time: 10 min
Reaction terminator: 10 μL of 2 M sodium carbonate
Absorbance: 405 nm

### Conditions for Determination of β-Xylosidase Specific Activity

Substrate: p-nitrophenyl-β-xylopyranoside (produced by Sigma-Aldrich Japan)
Reaction solution: 90 μL of 50 mM acetate buffer containing 1 mM p-nitrophenyl-β-xylopyranoside
Enzyme dilution: 10 μL
Reaction temperature: 30° C.
Reaction time: 10 min
Reaction terminator: 10 μL of 2 M sodium carbonate
Absorbance: 405 nm

### Conditions for Determination of Cellobiohydrolase Specific Activity

Substrate: p-nitrophenyl-β-lactopyranoside (produced by Sigma-Aldrich Japan)
Reaction solution: 90 μL of 50 mM acetate buffer containing 1 mM p-nitrophenyl-β-lactopyranoside
Enzyme dilution: 10 μL
Reaction temperature: 30° C.
Reaction time: 10 min
Reaction terminator: 10 μL of 2 M sodium carbonate
Absorbance: 405 nm

### Reference Example 3: Saccharification Test of Cellulose-Containing Biomass

As cellulose-containing biomass, use was made of Arbocel (registered trademark) B800 (produced by J. Rettenmaier & Sohne) or bagasse powdered to an average particle diameter of 100 μm. As an enzyme solution, use was made of a filtrate obtained by collecting a 1 mL portion of a culture solution of either a Trichoderma reesei mutant strain or the Trichoderma reesei parent strain into which the mutation had not been introduced, centrifuging the collected culture solution, recovering a supernatant from which the fungus bodies had been removed, and filtering the supernatant with a 0.22-μm filter.

### Saccharification Reaction

100 μL of a 1 M sodium acetate buffer was used as a buffer for saccharification reaction; 2 μL of 50 g/L erythromycin solution was used to prevent the propagation of various germs; and 0.1 g of Arbocel (registered trademark) B800 (produced by J. Rettenmaier & Sohne) or bagasse powdered to an average particle diameter of 100 μm was used as a material to be saccharified. Enzyme solutions were prepared in the following manner. An enzyme solution obtained by flask cultivation using Arbocel (registered trademark) B800 was introduced in an amount of 150 μL into a measuring cylinder. An enzyme solution obtained by flask cultivation using lactose was introduced in an amount of 300 μL into a measuring cylinder. An enzyme solution obtained by 5 L jar fermenter cultivation was introduced into a measuring cylinder in such an amount as to result in a protein concentration of 0.8 mg. The enzyme solution in each measuring cylinder was diluted with sterilized water to 1 mL in total, and the dilution introduced into a 2 mL tube. A saccharification reaction was conducted under temperature conditions of 50° C. for 24 hours, and the saccharification mixture centrifuged. The resultant supernatant was recovered as a saccharified solution, and the enzymatic reaction terminated by adding 1 N NaOH solution in an amount of one-tenth the amount of the recovered saccharified solution. The glucose concentration in the saccharified solution after the termination of the reaction was determined by the UPLC shown below.

### Determination of Glucose Concentration

Glucose was quantitatively analyzed under the following conditions using ACQUITY UPLC System (Waters). The quantitative analysis was performed on the basis of a calibration curve drawn with standard solutions of glucose.
Column: ACQUITY UPLC BEH Amide 1.7 μm 2.1×100 mm Column
Separation method: HILIC

Mobile phase: mobile phase A: 80% acetonitrile, 0.2% aqueous TEA solution, and mobile phase B: 30% acetonitrile, 0.2% aqueous TEA solution, in accordance with the following gradient. The gradient was a linear gradient reaching the mixing ratio corresponding to the time below.

Initiation condition: (A 99.90%, B 0.10%), 2 minutes after initiation: (A 96.70%, B 3.30%), 3.5 minutes after initiation: (A 95.00%, B 5.00%), 3.55 minutes after initiation: (A 99.90%, B 0.10%), 6 minutes after initiation: (A 99.90%, B 0.10%)
Detection method: ELSD (evaporative light scattering detector)
Flow rate: 0.3 mL/min
Temperature: 55° C.

### Example 1

Preparation of Trichoderma reesei Mutant Strain in which the Function of Polypeptide Consisting of the Amino Acid Sequence Represented by SEQ ID NO: 4 has been Eliminated

### Method of Preparing Mutant Strain

A Trichoderma reesei mutant strain in which the function of the polypeptide consisting of the amino acid sequence represented by SEQ ID NO: 4 has been eliminated is prepared in the following manner. A gene represented by SEQ ID NO: 1 that encodes the polypeptide consisting of the amino acid sequence represented by SEQ ID NO: 4 is destroyed by replacing the gene with acetamide as a selection marker and with acetamidase (AmdS) gene (amdS) capable of decomposing acetamide as a selection marker gene. A DNA fragment consisting of the gene sequence represented by SEQ ID NO: 13 is prepared to eliminate the function of the polypeptide consisting of the amino acid sequence represented by SEQ ID NO: 4, and Trichoderma reesei ATCC66589 strain is transformed with the DNA fragment, thereby preparing the Trichoderma reesei mutant strain in which the function of the polypeptide consisting of the amino acid sequence represented by SEQ ID NO: 4 has been eliminated. By this method, a Trichoderma reesei mutant strain is obtained in which the base sequence represented by SEQ ID NO: 1 has been deleted. To allow a DNA fragment consisting of the base sequence represented by SEQ ID NO: 1 to be introduced upstream and downstream an amdS-containing DNA sequence, a plasmid for mutation introduction is prepared to add a portion homologous to the gene sequence of the Trichoderma reesei QM9414 strain.

Specifically, PCR is conducted using genomic DNA extracted in a usual manner from the Trichoderma reesei QM9414 strain and oligo DNAs represented by SEQ ID NOs: 14 and 15, and the resulting amplified fragment is treated with restriction enzymes AflII and NotI to obtain a DNA fragment for use as the upstream DNA fragment. In addition, PCR is conducted using oligo DNAs represented by SEQ ID NOs: 16 and 17, and the resulting amplified fragment is treated with restriction enzymes MluI and SpeI to obtain a DNA fragment for use as the downstream DNA fragment. The upstream and downstream DNA fragments are introduced into a plasmid to which amdS has been inserted by using restriction enzymes AflII and NotI and restriction enzymes MluI and SpeI, respectively, to construct a plasmid for mutation introduction. The plasmid for mutation introduction is treated with restriction enzymes AflII and SpeI, and the Trichoderma reesei ATCC66589 strain is transformed with the obtained DNA fragment which is shown by SEQ ID NO: 13. The manipulations involving the molecular biological technique are performed as described in Molecular cloning, laboratory manual, 1st, 2nd, 3rd (1989). In addition, the transformation is carried out using a standard technique, i.e., a protoplast PEG method, and specifically, is performed as described in Gene, 61, 165-176 (1987).

### Preparation and Evaluation of the Mutant Strain

The Trichoderma reesei mutant strain obtained by the method described above was used as Trichoderma reesei mutant strain I in the following protein production test and experiments to determine protein concentration and cellulase specific activity.

### Example 2

Preparation of Trichoderma reesei Mutant Strain in which the Function of Polypeptide Consisting of the Amino Acid Sequence Represented by SEQ ID NO: 5 has been Eliminated

### Method of Preparing Mutant Strain

A Trichoderma reesei mutant strain in which the function of the polypeptide consisting of the amino acid sequence represented by SEQ ID NO: 5 has been eliminated is prepared in the following manner. A gene represented by SEQ ID NO: 2 that encodes the polypeptide consisting of the amino acid sequence represented by SEQ ID NO: 5 is destroyed by replacing the gene with acetamide as a selection marker and with acetamidase (AmdS) gene (amdS) capable of decomposing acetamide as a selection marker gene. A DNA fragment consisting of the gene sequence represented by SEQ ID NO: 18 is prepared to eliminate the function of the polypeptide consisting of the amino acid sequence represented by SEQ ID NO: 5, and Trichoderma reesei ATCC66589 strain is transformed with the DNA fragment, thereby preparing the Trichoderma reesei mutant strain in which the function of the polypeptide consisting of the amino acid sequence represented by SEQ ID NO: 5 has been eliminated. By this method, a Trichoderma reesei mutant strain is obtained in which the base sequence represented by SEQ ID NO: 2 has been deleted. To allow a DNA fragment consisting of the base sequence represented by SEQ ID NO: 2 to be introduced upstream and downstream an amdS-containing DNA sequence, a plasmid for mutation introduction is prepared to add a portion homologous to the gene sequence of the Trichoderma reesei QM9414 strain.

Specifically, a synthesized DNA fragment shown by SEQ ID NO: 19 is treated with restriction enzymes AflII and NotI to obtain a DNA fragment for use as the upstream DNA fragment. In addition, PCR is conducted using genomic DNA extracted in a usual manner from the Trichoderma reesei QM9414 strain and oligo DNAs represented by SEQ ID NOs: 20 and 21, and the resulting amplified fragment is treated with restriction enzymes MluI and SpeI to obtain a DNA fragment for use as the downstream DNA fragment. The upstream and downstream DNA fragments are introduced into a plasmid to which amdS has been inserted by using restriction enzymes AflII and NotI and restriction enzymes MluI and SpeI, respectively, to construct a plasmid for mutation introduction. The plasmid for mutation introduction is treated with restriction enzymes AflII and SpeI, and the Trichoderma reesei ATCC66589 strain is transformed with the obtained DNA fragment which is shown by SEQ ID NO: 18. The manipulations involving the molecular biological technique are performed as described in Molecular cloning, laboratory manual, 1st, 2nd, 3rd (1989). In addition, the transformation is carried out using a standard technique, i.e., a protoplast PEG method, and specifically, is performed as described in Gene, 61, 165-176 (1987).

### Preparation and Evaluation of the Mutant Strain

The Trichoderma reesei mutant strain obtained by the method described above was used as Trichoderma reesei mutant strain II in the following protein production test and experiments to determine protein concentration and cellulase specific activity.

### Example 3

Preparation of Trichoderma reesei Mutant Strain in which the Function of Polypeptide Consisting of the Amino Acid Sequence Represented by SEQ ID NO: 6 has been Eliminated

### Method of Preparing Mutant Strain

A Trichoderma reesei mutant strain in which the function of the polypeptide consisting of the amino acid sequence represented by SEQ ID NO: 6 has been eliminated is prepared in the following manner. A gene represented by SEQ ID NO: 3 that encodes the polypeptide consisting of the amino acid sequence represented by SEQ ID NO: 6 is destroyed by replacing the gene with acetamide as a selection marker and with acetamidase (AmdS) gene (amdS) capable of decomposing acetamide as a selection marker gene. A DNA fragment consisting of the gene sequence represented by SEQ ID NO: 22 is prepared to eliminate the function of the polypeptide consisting of the amino acid sequence represented by SEQ ID NO: 6, and Trichoderma reesei ATCC66589 strain is transformed with the DNA fragment, thereby preparing the Trichoderma reesei mutant strain in which the function of the polypeptide consisting of the amino acid sequence represented by SEQ ID NO: 6 has been eliminated. By this method, a Trichoderma reesei mutant strain is obtained in which the base sequence represented by SEQ ID NO: 3 has been deleted. To allow a DNA fragment consisting of the base sequence represented by SEQ ID NO: 3 to be introduced upstream and downstream an amdS-containing DNA sequence, a plasmid for mutation introduction is prepared to add a portion homologous to the gene sequence of the Trichoderma reesei QM9414 strain.

Specifically, PCR is conducted using genomic DNA extracted in a usual manner from the Trichoderma reesei QM9414 strain and oligo DNAs represented by SEQ ID NOs: 23 and 24, and the resulting amplified fragment is treated with restriction enzymes AflII and NotI to obtain a DNA fragment for use as the upstream DNA fragment. In addition, PCR is conducted using oligo DNAs represented by SEQ ID NOs: 25 and 26, and the resulting amplified fragment is treated with restriction enzymes MluI and XhoI to obtain a DNA fragment for use as the downstream DNA fragment. The upstream and downstream DNA fragments are introduced into a plasmid to which amdS has been inserted by using restriction enzymes AflII and NotI and restriction enzymes MluI and XhoI, respectively, to construct a plasmid for mutation introduction. The plasmid for mutation introduction is treated with restriction enzymes AflII and SpeI, and the Trichoderma reesei ATCC66589 strain is transformed with the obtained DNA fragment which is shown by SEQ ID NO: 22. The manipulations involving the molecular biological technique are performed as described in Molecular cloning, laboratory manual, 1st, 2nd, 3rd (1989). In addition, the transformation is carried out using a standard technique, i.e., a protoplast PEG method, and specifically, is performed as described in Gene, 61, 165-176 (1987).

### Preparation and Evaluation of the Mutant Strain

The Trichoderma reesei mutant strain obtained by the method described above was used as Trichoderma reesei mutant strain III in the following protein production test and experiments for determining protein concentration and cellulase specific activity.

### Example 4

Protein Production Test Using Trichoderma reesei Mutant Strains

### Preculture

After spores of each of the Trichoderma reesei mutant strains prepared in Examples 1 to 3 are diluted with physiological saline to be 1.0×10⁷/mL, 2.5 mL of the diluted spore solution is inoculated into 250 mL of the preculture medium shown in Table 1 that has been placed in a 1 L baffled flask, and incubated on a shaker under the conditions of 28° C. and 120 rpm for 72 hours. Trichoderma reesei ATCC66589 strain is used as a control to conduct the same experiments shown below.

TABLE 1

| | | |
|---|---|---|
| Glucose | 20 | g |
| 5× Mandel's solution* | 200 | mL |
| 10× Ammonium tartrate solution** | 100 | mL |
| Corn steep liquor | 50 | g |
| Trace element solution*** | 1 | mL |
| Tween 80 | 0.5 | mL |
| PE-M | 1 | mL |

(per 1 L)

```
*The 5× Mandel's solution has the following composition.
  7 g/L (NH₄)₂SO₄
10 g/L KH₂PO₄
  2 g/L CaCl₂•2H₂O
1.5 g/L MgSO₄•7H₂O
**The 10× Ammonium tartrate solution contains 92 g/L ammonium tartrate.
***The trace element solution has the following composition.
0.3 g/L H₃BO₃
1.3 g/L (NH₄)₆Mo₇O₂₄•4H₂O
  5 g/L FeCl₃•6H₂O
  2 g/L CuSO₄•5H₂O
0.4 g/L MnCl₂•4H₂O
10 g/L ZnCl₂
```

### Main Culture

Arbocel (registered trademark) B800 is added to the main-culture medium shown in Table 2, and an investigation of submerged culture is conducted using a 5 L jar fermenter (manufactured by ABLE & Biott Co., Ltd.).

The preculture solutions of the Trichoderma reesei ATCC66589 strain and the Trichoderma reesei mutant strains prepared in Examples 1, 2, and 3 are each inoculated in an amount of 200 mL into 2 L of the main-culture medium to which Arbocel (registered tradename) B800 has been added.

After inoculation of each preculture medium into the main-culture medium, submerged culture is performed under the cultivation conditions of 28° C., 700 rpm, and an air flow rate of 100 mL/min while regulating the pH to 5.0.

TABLE 2

| | | |
|---|---|---|
| Arbocel B800 (produced by | 100 | g |
| J. Rettenmaier & Sohne) | | |
| 5× Mandel's solution* | 200 | mL |
| Corn steep liquor | 25 | g |
| Trace element solution*** | 1 | mL |
| Tween 80 | 0.5 | mL |
| PE-M | 1 | mL |

(per 1 L)

*Same as in Table 1.
***Same as in Table 1.

### Addition of Liquid Sugar During Main Culture

At 40 hours after initiation of the main culture, the liquid sugar culture medium shown in Table 3 is continuously added to the main-culture solution in an amount of 250 mL per day.

TABLE 3

| | |
|---|---|
| Glucose | 500 g |
| Lactose | 125 g |
| (NH₄)₂SO₄ | 8.75 g |
| | (per 1 L) |

### Collection of Culture Solutions

At each of given time periods after initiation of the cultivation, 20 mL portion of each of the main-culture solutions of the Trichoderma reesei mutant strains prepared in Examples 1 to 3 is collected. A part of the collected culture solution is centrifuged under the conditions of 15,000×g and 4° C. for 10 minutes to obtain a supernatant. The supernatant is filtered with a 0.22 μm filter, and the filtrate is used as a cellulase solution in the following experiments.

### Determination of Protein Concentration

The protein concentration of each of the culture solutions of Examples 1 to 3 which have been collected at intervals after initiation of the cultivation is determined under the conditions shown in Reference Example 1. As a result, the Trichoderma reesei mutant strains prepared in Examples 1 to 3 give culture solutions having higher protein concentrations, in terms of relative value, than that of the Trichoderma reesei ATCC66589 strain.

### Determination of Enzyme Activities

The culture solutions of Examples 1 to 3 that have been collected at intervals after initiation of the cultivation are used as enzyme solutions to determine the specific activities of β-glycosidase, β-xylosidase, and cellobiohydrolase under the conditions shown in Reference Example 2. In determining the specific activity, an increase in absorbance at 405 nm is measured, and release of 1 μmol of the substrate per minute is defined as 1 U of activity to calculate the specific activity. As a result, the culture solutions of the Trichoderma reesei mutant strains prepared in Examples 1 to 3 are higher in the three specific activities than the culture solution of the Trichoderma reesei ATCC66589 strain.

### Flask Cultivation

Spores of each of the Trichoderma reesei mutant strains I to III prepared in Examples 1 to 3 were diluted with physiological saline to be 1.0×10⁷/mL, and 0.1 mL of the resultant spore dilution was inoculated into 10 mL of the flask culture medium containing Arbocel (registered trademark) B800 or lactose shown in Table 4, that had been placed in a 50 mL baffled flask. This spore dilution was incubated on a shaker under the conditions of 28° C. and 120 rpm for 120 hours.

Furthermore, the Trichoderma reesei ATCC66589 strain, which was the parent strain into which any of the mutations of mutant strains I to III had not been introduced, was subjected to 120 hours incubation by the method shown above, as a control for the mutant strains.

TABLE 4

| | | |
|---|---|---|
| Arbocel B800 (produced by J. | 20 | g |
| Rettenmaier & Sohne) Or | | |
| Lactose (produced by | 20 | g |
| Kanto Chemical Co., Inc.) | | |
| 5× Mandel's solution* | 200 | mL |
| 10× Ammonium tartrate solution** | 100 | mL |
| Corn steep liquor | 50 | g |
| Trace element solution*** | 1 | mL |
| Tween 80 | 0.5 | mL |
| PE-M | 1 | mL |

(per 1 L)

*Same as in Table 1.
**Same as in Table 1.
***Same as in Table 1.

### Collection of Culture Solutions

At 120 hours after initiation of the flask cultivation, 1 mL portion of each culture solution was collected. The culture solution was centrifuged under the conditions of 15,000×g and 4° C. for 10 minutes to obtain a supernatant. The supernatant was filtered with a 0.22 μm filter, and the filtrate was used in the following experiments.

### Determination of Protein Concentration

In the flask cultivation using Arbocel (registered trademark) B800, when the protein concentration in the culture solution obtained by cultivating the Trichoderma reesei ATCC66589 strain was taken as 1, then the relative values of the protein concentrations in the culture solutions of Trichoderma reesei mutant strains I, II, and III were 1.1 each. It was thus ascertained that the mutant strains had a higher protein-producing ability than the parent strain.

Also in the flask cultivation using lactose, when the protein concentration in the culture solution obtained by cultivating the Trichoderma reesei ATCC66589 strain was taken as 1, then the protein concentration for Trichoderma reesei mutant strain I was 1.2, for mutant strain II was 1.3, and for mutant strain III was 1.2. It was thus ascertained that the mutant strains had a higher protein-producing ability than the parent strain.

### Determination of Various Cellulase Specific Activities

In the flask cultivation using Arbocel (registered trademark) B800, when various cellulase specific activities of the culture solution obtained by cultivating the Trichoderma reesei ATCC66589 strain were taken as 1, then the β-glucosidase specific activities were 1.1 for Trichoderma reesei mutant strain I, 1.2 for mutant strain II, and 1.1 for mutant strain III, the β-xylosidase specific activity was 1.1 for all the Trichoderma reesei mutant strains I, II, and III, and the cellobiohydrolase specific activity was also 1.1 for all the mutant strains I, II, and III. It was thus ascertained that the mutant strains had the unexpected effect of bringing about improvements in various cellulase specific activities.

Also in the flask cultivation using lactose, when various cellulase specific activities of the culture solution obtained by cultivating the Trichoderma reesei ATCC66589 strain were taken as 1, then the β-glucosidase specific activities were 1.1 for Trichoderma reesei mutant strain I, 1.2 for mutant strain II, and 1.4 for mutant strain III, the β-xylosidase specific activities were 1.1 for Trichoderma reesei mutant strain I, 1.4 for mutant strain II, and 1.4 for mutant strain III, and the cellobiohydrolase specific activities were 1.2 for mutant strain I, 1.1 for mutant strain II, and 1.1 for mutant strain III. It was thus ascertained that the mutant strains had the unexpected effect of bringing about improvements in various cellulase specific activities.

### Saccharification Reaction Test

In accordance with the technique described in Reference Example 3, culture solutions collected at 120 hours after initiation of the flask cultivation of Trichoderma reesei mutant strains I, II, and III were used as cellulases to conduct a saccharification reaction test of cellulose-containing biomass. As the cellulose-containing biomass, Arbocel (registered trademark) B800 or powdered bagasse was used.

As a result, in the saccharification reaction for saccharifying Arbocel (registered trademark) B800, when the glucose concentration in the saccharified solution obtained using the cellulases obtained from the Trichoderma reesei ATCC66589 strain by the flask cultivation using lactose was taken as 1, then the relative value of the glucose concentration in the saccharified solution obtained using the cellulases obtained from Trichoderma reesei mutant strain I was 1.2, the relative value of the glucose concentration in the saccharified solution obtained using the cellulases obtained from mutant strain II was 1.3, and the relative value of the glucose concentration in the saccharified solution obtained using the cellulases obtained from mutant strain III was 1.1. Meanwhile, when the glucose concentration in the saccharified solution obtained using the cellulases obtained from Trichoderma reesei ATCC66589 strain by the flask cultivation using Arbocel (registered trademark) B800 was taken as 1, then the relative value of the glucose concentration in the saccharified solution obtained using the cellulases obtained from Trichoderma reesei mutant strain I was 1.1, the relative value of the glucose concentration in the saccharified solution obtained using the cellulases obtained from mutant strain II was 1.2, and the relative value of the glucose concentration in the saccharified solution obtained using the cellulases obtained from mutant strain III was 1.1.

In the saccharification reaction for saccharifying powdered bagasse, when the glucose concentration in the saccharified solution obtained using the cellulases obtained from the Trichoderma reesei ATCC66589 strain by the flask cultivation using lactose was taken as 1, then the relative value of the glucose concentration in the saccharified solution obtained using the cellulases obtained from Trichoderma reesei mutant strain I was 1.3, the relative value of the glucose concentration in the saccharified solution obtained using the cellulases obtained from mutant strain II was 1.3, and the relative value of the glucose concentration in the saccharified solution obtained using the cellulases obtained from mutant strain III was 1.1. Meanwhile, when the glucose concentration in the saccharified solution obtained using the cellulases obtained from Trichoderma reesei ATCC66589 strain by the flask cultivation using Arbocel (registered trademark) B800 was taken as 1, then the relative value of the glucose concentration in the saccharified solution obtained using the cellulases obtained from Trichoderma reesei mutant strain I was 1.2, the relative value of the glucose concentration in the saccharified solution obtained using the cellulases obtained from mutant strain II was 1.3, and the relative value of the glucose concentration in the saccharified solution obtained using the cellulases obtained from mutant strain III was 1.1.

It was ascertained from those results that the cellulases produced by each of the Trichoderma reesei mutant strains I to III were superior in enzymatic activity to the cellulases produced by the parent strain and hence had an excellent ability to produce glucose from cellulose-containing biomass.

### Example 5

### Preparation of Trichoderma reesei Mutant Strain in which the Functions of Polypeptides Consisting of Amino Acid Sequences Represented by SEQ ID NOs: 4, 5, and 6 have been Eliminated

A QM9414-A strain, which was a strain obtained by passage culture of Trichoderma reesei QM9414 strain, was subjected to a genetic mutation treatment to acquire a QM9414-B strain as a mutant strain. The genetic mutation treatment was conducted in the following manner. Spores of the QM9414-A strain were inoculated into the preculture medium shown in Table 1 so that 1.0×10⁵ spores were inoculated per mL of the preculture medium. 15 mL of the preculture medium was incubated for a half day and then centrifuged to recover the spores. The recovered spores were suspended in a Tris-maleate buffer (pH 6.0) to give a 10 mL spore solution, and 0.5 mL of an NTG solution obtained by dissolution with a Tris-maleate buffer (pH 6.0) to result in a concentration of 1.0 g/L was added thereto. The resultant mixture was held at 28° C. for 100 minutes to perform the genetic mutation treatment. The spores that had undergone the genetic mutation treatment were recovered by centrifuging, subsequently rinsed with a Tris-maleate buffer (pH 6.0) three times, and finally suspended as genetic-mutation-treated spores in 10 mL of a Tris-maleate buffer (pH 6.0). Subsequently, the genetic-mutation-treated spores were added to an agar medium prepared by adding crystalline cellulose. The size of halos that surrounded colonies and indicated regions where the crystalline cellulose had been decomposed by cellulases was used as an index to select a QM9414-B strain which had formed a large halo.

The QM9414-B strain was genetically analyzed and, as a result, was ascertained to have undergone the following mutations in the base sequences represented by SEQ ID NOs: 1, 2, and 3.

In the base sequence represented by SEQ ID NO: 1, one guanine base residue had been inserted into the 85th position. This mutation changed the 30th amino acid residue from the N-terminal side in the amino acid sequence represented by SEQ ID NO: 4 from histidine to threonine, and the succeeding frameshifts caused the translation to end at the 90th amino acid residue from the N-terminal side.

In the base sequence represented by SEQ ID NO: 2, 46 base residues represented by SEQ ID NO: 27 had been inserted into the 6th position. This mutation changed the glutamine residue at the 3rd residue from the N-terminal side in the amino acid sequence represented by SEQ ID NO: 5 into arginine and caused the translation to end at that position.

In the base sequence represented by SEQ ID NO: 3, the one cytosine base residue at the 499th residue had been deleted. This mutation changed the 167th amino acid residue from the N-terminal side in the amino acid sequence represented by SEQ ID NO: 6 from alanine to arginine, and the succeeding frameshifts caused the translation to end at the 193rd position from the N-terminal side.

### Example 6

Protein Production Test Using Trichoderma reesei QM9414-B Strain

The mutant strain QM9414-B strain obtained in Example 5 was cultivated in accordance with the (Preculture), (Main Culture), (Addition of Liquid Sugar during Main Culture), and (Collection of Culture Solutions) described in Example 4, and the protein concentration was determined under the conditions shown in Reference Example 1. The parent strain QM9414-A strain was used as a control and cultivated in the same manner as for the QM9414-B strain, and the protein concentration was determined under the conditions shown in Reference Example 1.

The produced-protein concentrations at 120 hours after initiation of the cultivation were determined and, as a result, the QM9414-B strain gave an increased relative value which was 1.2 times higher than that of the QM9414-A strain. Likewise, the protein concentrations at 200 hours after initiation of the cultivation were determined. As a result, the QM9414-A strain gave a protein concentration which was unchanged from that obtained at 120 hours after initiation of the cultivation, whereas the QM9414-B strain gave an increased protein concentration which was 1.3 times higher than the protein concentration obtained at 120 hours after initiation of the cultivation.

When the protein concentration given by the QM9414-A strain at 120 hours after initiation of the cultivation was taken as 1, the relative value of the protein concentration given by the QM9414-B strain was 1.8. When the protein concentration given by the QM9414-A strain at 185 hours after initiation of the cultivation was taken as 1, the relative value of the protein concentration given by the QM9414-B strain was 2.7.

What is claimed:

1. A mutant strain of Trichoderma reesei, the mutant strain having a mutation that eliminates an expression of a polypeptide consisting of the amino acid sequence of SEQ ID NO: 6.

2. The mutant strain according to claim 1, wherein the mutation is a mutation of an amino acid sequence of an F-box domain region of the polypeptide consisting of the amino acid sequence represented by SEQ ID NO: 6.

3. The mutant strain according to claim 2, wherein the mutation is a deletion of the polypeptide consisting of the amino acid sequence represented by SEQ ID NO: 6, the deletion being caused by a frameshift mutation accompanying the mutation of the amino acid sequence of the F-box domain region.

4. The mutant strain of claim 3, wherein the mutation is a frameshift mutation due to a mutation in which an alanine residue at the 167th residue from the N-terminal side in the amino acid sequence represented by SEQ ID NO: 6 is changed to a residue of an amino acid other than alanine.

5. The mutant strain of claim 1, wherein the mutant strain has a mutation in a gene encoding a polypeptide consisting of an amino acid sequence of SEQ ID NO: 6 that eliminates an expression of the polypeptide consisting of the amino acid sequence of SEQ ID NO: 6.

6. The mutant strain of claim 5, wherein the mutation results in the deletion of the gene encoding the polypeptide consisting of the amino acid sequence of SEQ ID NO: 6.

7. A method of producing a protein, the method comprising a step of cultivating the mutant strain according to claim 1.

8. The method of producing a protein according to claim 7, wherein, in the cultivation step, at least one inducer selected from the group consisting of lactose and glucose is added to a culture medium in the middle of a cultivation.

9. A method of producing a protein, the method comprising a step of cultivating the mutant strain according to claim 1, in a culture medium at least comprising lactose.

10. A method of producing a cellulase, the method comprising a step of cultivating the mutant strain according to claim 1.

11. The method of producing a cellulase according to claim 10, wherein, in the cultivation step, at least one inducer selected from the group consisting of lactose and glucose is added to a culture medium in the middle of a cultivation.

12. A method of producing a cellulase, the method comprising a step of cultivating the mutant strain according to claim 1, in a culture medium at least comprising lactose.

13. A method of producing a sugar, the method comprising:
a step of producing a cellulase by the method of producing a cellulase according to claim 11; and
a step of saccharifying a cellulose-containing biomass using the cellulase obtained in the step.

14. A method of producing a protein, the method comprising a step of cultivating the mutant strain according to claim 6.

15. The method of producing a protein according to claim 14, wherein, in the cultivation step, at least one inducer selected from the group consisting of lactose and glucose is added to a culture medium in the middle of a cultivation.

16. A method of producing a protein, the method comprising a step of cultivating the mutant strain according to claim 6, in a culture medium at least comprising lactose.

17. A method of producing a cellulase, the method comprising a step of cultivating the mutant strain according to claim 6.

18. The method of producing a cellulase according to claim 17, wherein, in the cultivation step, at least one inducer selected from the group consisting of lactose and glucose is added to a culture medium in the middle of a cultivation.

19. A method of producing a cellulase, the method comprising a step of cultivating the mutant strain according to claim 6, in a culture medium at least comprising lactose.

20. A method of producing a sugar, the method comprising:
a step of producing a cellulase by the method of producing a cellulase according to claim 18; and
a step of saccharifying a cellulose-containing biomass using the cellulase obtained in the step.


SEQUENCE LISTING (27 sequences)

SEQ ID NO: 1 - DNA, 1975 bp, Trichoderma reesei
  source 1..1975: mol_type = unassigned DNA
cctaccatcagcatgtctccaccgagtcccgtcgaccatccgatggagggcacgtcgccagaggacaactcgcccacgggccacggccacggctccggctccgaccccgaaggcagctccaatggcaagccgcccgtctcccgggccggcaacgcatcgccgcaggagcacggcgccctcaccacctcgagcaacatgcccgcccctcctcccgccgccgccgctgctgttcaccagcccaagatcgtccagactgcctttatccataagctatacaagtgaggaccctggcctccctctctcccccaaacccccctttcccccttcaatgcttccttgacctgccaaggagggccccgagactaaagcgacagctgctaacggcgcaaatagcatgttggaagacaccagtatacagcatctcatatcctggtcatcgtccgccgaaagcttcgtcatgtcgccctctgccgacttctccaaggtattatcgtaagcagtgccccgacaatcctcgcccttttctggccaattcgctcattctcgcccccagacaatacttcaaacacaccaacatatcgtctttcgtgcgtcagctcaacatgtacggattccataaaggtacctcctgcccccgcttgcttctgcctcgaacctccgaacctcgaatggcttaccatggatgcagaacgagacgtgtttcataccggcaaccccgaaacgacgctctgggaattcaagcacggcaacggcaacttcaagcgcggcgacctcgtcggccttcgagagatcaagcgtcgcgccagccgacacgctctcgtcaaccgggaaaacacctttcccaagacctccacatctcagcctggcacgcccattgagcctgttcaagtcccgccggatagcatcgaggctagaatagccaacctggagcactccctgtacgatacggctgcgagactgcagagaagcgaggaatcggcccactacatgcatgtcaagaaccaggccatcatggagacactcaaccgactgctcttcttcaaccaagagttgtccaaggggatactgtcgcttgtacctccagacaaccctgtacacagagatggtttgtcacacgaaacaatcaggacgatacgctattcatgggagacgatgaaactgacaatgagggcttcaagtcatgacactccagggcgaaatggtgaggcaggcagaaatgctgcgctcgctcgacgagccgcacgagcctgtctattccgccaggcaacagttcttcggcacggtcgacaacgcccccgtgtccccgcggcagcttcctcaagacgacaacaggcgagtcacgctcaacgttccgcagggccggagtcagccgtcgtaccgaccagccgtcccctcgaacctgtccgccggcacgcggcgaccctacggatccatcagcggcggtgccggctcctcacctctgcgcaatgccgcgccggcacccccagcagggccgcatcccctttccaacgtggagaccgtccccagcaacctggccagacgccataccgccgcagacattcgcgcacacggatggcagcccaacgcaccgccttatccccctggagccgtgccgccgcccgtatggcctcaatctcccaatcggcccgaggatcagaggataagagactctctctcttcgttttccctgcaggccccatcggctcatggacacccgcactcgcggccggcgacgccaccccacgctcccttttcaaacggcgcgagcggagggtcggatacctttggcagctggtcgtggggcgcggcatcgaaccgcgagagcaagacgattggagcgttgaaggagtcgtcagcgccgcccaccaggaggggcagcatggcacacatcctcaaccccagcgataccgctgagcgatcagacgaggacgaggacccccggggcgacgacgacagaaagcgaaaacgaagacag

SEQ ID NO: 2 - DNA, 1900 bp, Trichoderma reesei
  source 1..1900: mol_type = unassigned DNA
atgggccaagccccttctcagacgcggcggactcggcggacgcacgaggaactgacacaggagcttgtgagcgcgccgttgcaccttggcctgggaacaagcaagggcctaggctttgctaactgtgcgtgaacaggcgtacagattcaaggaaaagtgcttcacgtcgctggagtactactcactgaaggatgtcttcaagaaactggccgaccagcagggcgacatacggtatctcaaggaggacaccatagctcgcttcctcgagatcccagacatcctcggggcttcgccggtcatcttccacatgatctcgtatctgggtgcgtttccctttctgcaggaggcccccgtggtgctcgagctggcgcagttgatcatggtcgttgtcatcatgacggagcgatataagcgcgttcttgcgaagggctcgacagacaggacgaaattattttttaaaagcttggctgtgtatgaccggaaagtgatggaggagaccggttcttcaccgcggaattccacttcaaaagacgccactgcgaggccgagcgccaacacaagaggctttgcaatcgacgagccgatggccgaggacgaggatgatggcgacgacgacgacgatgatcttgtcattacggccttcgagctgctcgacatcgatgaagctaccaagcacggagaagccgctgccattaaagaagccatcatcccgaccgacaacttccgcaaacttattatgctgctgctgctgattgcgccactggatgctcaggagagtctatcgcaatattccagccgggtcgcgggccccgaactagagtcgctgcgagccacggcagagtgtgtactagcatccttcgtcgatgtcgagacgtcgccaggcattggatatacccggttcaagactgtcattccggtcctattccccaacctctttgctgggttcaatggcctgtttgaacactttcttttctccaaggatctggacttctcgaagcacaaggttgagaagcctggtgacgagcagctcatcattggcaagattgcgcaacctctgctcccaacccctggcgacatcatgactgagcatacgctgtcgcaactttcactgtttctacctggctcctctctgttccgaagagtgagattgctctattcgggaaacgatgctggattctccatgggcagcctccagaccaaggtctttaactggagagccccaaccattcttctagtcagcggatcgagattagcagacgtccccgagggaggccaagaggcatcgttcgcctcttcgcttcccaccaaacggttccctcatggtagtaaatccgagcgtgtaacgtttggcgtgtacgttcgagagccttggaagcacacgcacaaagagtgcttcggcaattcggaaacaatactctttcaactagaacccattcacgatgttttccctgcctctacaatcaatacagactacgtcaccttcacgaaaccacctggcaaccggccctgtctagcatttgggtgcccacaccccaaaccgacgcagtcgcatcgcaaggagggcatacatgctttaggagccgtgtctctgttgtttgatgaatctttcgagttcggagtcttcaatcatgactacaagtcgagagggggcgctttccacactagcatcgtgaggaaatatgattttcaggatcggtttcgaatcgagaacatggaagtctggggatgtggtggtgacgaggaggccaaggcgcaagcagagagatgggcttgggaagaacgtgaagcggaagcccgtcgcaggatcaaccttgggacgggtgacattgaggcggacagggcattgctggagatggccgggctagtaggagggaatcgcagcggcggttcaatgggttga

SEQ ID NO: 3 - DNA, 3545 bp, Trichoderma reesei
  source 1..3545: mol_type = unassigned DNA
atgacccaagagcctctcaagactccagcacagctcccggccaacaatcatctcgattccctccaactcccccagcatggctcccttcatcgcgacgccagtggccctggtcgaggccttcccggcttcagcctcggctcgcagcgccttcgcctcgacccggctgagcatcagggccgggaagccggagtgaacgctgaacggctggacgagaagctgcaacaattgagcctcgatggagcccacgacggtcgcccttctgtgcccggacagcgagtctacgagtatgagaaggcctcgactccccaagcctctaagcagggccttggattccaggtcatcaaacgctcagagccccacgccgatggactcagtctcgaggactttcccaatggttggttctgcctcttccttgcggcgagcgacccattgctgaccaatgactgccagagatcctcacccacatcttttctcatctgccccccgactgccactcgtcggtggcgctggtctcgaagcgattctgggcgctggtcaccacccatcatgcgtggcgcatggcctttatgcgcttcttcccgggacacaccatcctggagaacaacggcaaggctgccgccgccactcattccatgactgggccatcctccgacgttgtgcgtttcgagacgcgatacttccctcgactcacgccgctggccacttggaggagcgagtatcttctacggactcgccacctaagaagcttggctcgcggcaaaccagggcctcccgccggtgggagtgtgtctggccgcgtcggtcgcggtgggaagaagtcaagcgccgtcttgacctacaactcgaaactgccttggcaggtgacgaatctccacgccgtcttcctgaatggaaagaaaccgccccgcgttatgcaaggcgccggagatctcggtgcggcaaccattagcgaccccacgactggaaagattgagaaatggggcattgaggatctatacaccactcctcagctggacgaggttgcccccaacctggtgccttacggcttgggagacggcccagccggcgtccccaatgtcatggatgtgagctacacgtacggcatgatatctggggagggatttccaggtggccggccctactttcgaggggtcaatgaaatgcgtggtcgctacgttggcgcagagatcagtgccgtcgacactcatccggatattcccaagattcccgaaatgtccgatgcgatctgcagcgtctggattgccaagtcatcgaccgtcacggcgacgactcaatccatgtgcggcatgctcacaggctctgctctcggtattatcacggcctattcacttggctgggataccacgggcccgagatatgccaatggcgacgtcaccgcacgatgggtcgttagccctggtgttcccatcatatccctcaaggtcgatgatggcttcaaccagaagcgcaagtcgtcctccagggtatgggcggtggctttgaacgccctcggtgaggtttactacctgtgcgatgtacccgttggcaaacccgggcgaactactggcgaggacatgacgaggaatgcctggtatgctggccgtacagcctactggcaccttctcgaggcgactcgcagagttgctcgtgaagacgaggctgacaaaaacgcaactcgtggaggctattcgcctcgatcgccttctctcgacatgcatctcagcaaggaacagattgtggcagaggctcgggagattgaaaagtttatgcgctatcggccctcccactttcgaaaggtctgcgagggctgggacatgcagcggaagctggaggtcgactttgcgagtgatgatgggaaaggtgccggcgagagcatctttgtcatcgattgcggcctggcagagaaccgccctgttagcatccagcgctattcgcgttcattgatacccgttcagggcactccgagcgaatcctcgacaccacttgcgccggtcccgacatcgctgtttggcagcatcggaggctttgccaaccgcatttctcctgtttctgaatcccaagcgccattgtctccgccacctactcctaaatcgccccctgtgccttcgacggttcttcatgactggttcaaacaagggttcgagttcaagggccacggtcacgataccatattgtcagttgctttggacaattcgctcacctcgctctacacacttggggaggaccctctacacaccgcgaacgaagcgtcctccacgacgagcccctgggctgaacacggggcgagggagattcctgggcgccgaacccgcttcatcatcgctggaaccaactccggcgcggtgcttgtctggaatgcgcgtgatgacgatcgaactcgtgatatacaaccactgcgcatccttcagaccgagtcaccagaggtttctgctgtggcagcctctgggttgtatctcgttcacggcggcagcgacggccttgtccaagcctgggatcccttggcatctacaacggatcccatcagaacgattaacgctcggtcaaatggccgggtcccccgtcacatgctggtaatgaatcccgcattgcaggaggagacatactcggcagcaaaggccatatatcttgaccctgattctacgacgcttcaaggtgtagtctcttttggcgcattcctgcgatattggtcgtatgggtccaatggtcatgccacaggtcgcaagcggcgcgtccgacacgccgatatggacgctcggcttgcgagtcgccggcaaggccatgcagtgtcaggctacattgcctctgaggaagccgagatgcgacgggaggatgagcagcaggctcgcgagcacaaccgtcgtctcaagagatttggcgctctaggcgacttgaccgaggaagaagcgcttctctacgcccagatggtctcccaagaggcgtaccacgtagaggagcagcgacgggccagcgattcggcagccgacgccagcctggacaccgcctcttcctttagcgagaataccgtcgagactctgacacctgatccgagcgtcgccgatccggtcgcttcggaaacgagcggcatggccgaggatgacgagtacgagcagcagattcagcaggctatacgtctgtctctgctagaaggcgtcaacaacggcgtggaacagtcacctgtggattcctcacggggcaacagctctgttgatttcgaccaaccggtcaatgtcaagtacaagcccaagggcgggaagaaggggaagcaatcaggggcttcttctggtggctcaccgtctgcgagccacacgcccgttggtggtggtgcttcttcttcgcggctgagcacgactgaagatgaggatttagcgattgctctgagtctgagcatgcaggaccagggagggggatactcgccgccgggaatgtcgtcgtcgacgatgatggggaggagtgcttacttggaggctgctgctggtgtcgatgaggaggatgagtttccgtctttgcctggtgaagggaaggggaagggggtgcagagatggtga

SEQ ID NO: 4 - protein, 553 aa, Trichoderma reesei
PTISMSPPSPVDHPMEGTSPEDNSPTGHGHGSGSDPEGSSNGKPPVSRAGNASPQEHGALTTSSNMPAPPPAAAAAVHQPKIVQTAFIHKLYNMLEDTSIQHLISWSSSAESFVMSPSADFSKVLSQYFKHTNISSFVRQLNMYGFHKERDVFHTGNPETTLWEFKHGNGNFKRGDLVGLREIKRRASRHALVNRENTFPKTSTSQPGTPIEPVQVPPDSIEARIANLEHSLYDTAARLQRSEESAHYMHVKNQAIMETLNRLLFFNQELSKGILSLVPPDNPVHRDVMTLQGEMVRQAEMLRSLDEPHEPVYSARQQFFGTVDNAPVSPRQLPQDDNRRVTLNVPQGRSQPSYRPAVPSNLSAGTRRPYGSISGGAGSSPLRNAAPAPPAGPHPLSNVETVPSNLARRHTAADIRAHGWQPNAPPYPPGAVPPPVWPQSPNRPEDQRIRDSLSSFSLQAPSAHGHPHSRPATPPHAPFSNGASGGSDTFGSWSWGAASNRESKTIGALKESSAPPTRRGSMAHILNPSDTAERSDEDEDPRGDDDRKRKRRQ

SEQ ID NO: 5 - protein, 609 aa, Trichoderma reesei
MGQAPSQTRRTRRTHEELTQELAYRFKEKCFTSLEYYSLKDVFKKLADQQGDIRYLKEDTIARFLEIPDILGASPVIFHMISYLGAFPFLQEAPVVLELAQLIMVVVIMTERYKRVLAKGSTDRTKLFFKSLAVYDRKVMEETGSSPRNSTSKDATARPSANTRGFAIDEPMAEDEDDGDDDDDDLVITAFELLDIDEATKHGEAAAIKEAIIPTDNFRKLIMLLLLIAPLDAQESLSQYSSRVAGPELESLRATAECVLASFVDVETSPGIGYTRFKTVIPVLFPNLFAGFNGLFEHFLFSKDLDFSKHKVEKPGDEQLIIGKIAQPLLPTPGDIMTEHTLSQLSLFLPGSSLFRRVRLLYSGNDAGFSMGSLQTKVFNWRAPTILLVSGSRLADVPEGGQEASFASSLPTKRFPHGSKSERVTFGVYVREPWKHTHKECFGNSETILFQLEPIHDVFPASTINTDYVTFTKPPGNRPCLAFGCPHPKPTQSHRKEGIHALGAVSLLFDESFEFGVFNHDYKSRGGAFHTSIVRKYDFQDRFRIENMEVWGCGGDEEAKAQAERWAWEEREAEARRRINLGTGDIEADRALLEMAGLVGGNRSGGSMG

SEQ ID NO: 6 - protein, 1162 aa, Trichoderma reesei
MTQEPLKTPAQLPANNHLDSLQLPQHGSLHRDASGPGRGLPGFSLGSQRLRLDPAEHQGREAGVNAERLDEKLQQLSLDGAHDGRPSVPGQRVYEYEKASTPQASKQGLGFQVIKRSEPHADGLSLEDFPNEILTHIFSHLPPDCHSSVALVSKRFWALVTTHHAWRMAFMRFFPGHTILENNGKAAAATHSMTGPSSDVVRFETRYFPRLTPLATWRSEYLLRTRHLRSLARGKPGPPAGGSVSGRVGRGGKKSSAVLTYNSKLPWQVTNLHAVFLNGKKPPRVMQGAGDLGAATISDPTTGKIEKWGIEDLYTTPQLDEVAPNLVPYGLGDGPAGVPNVMDVSYTYGMISGEGFPGGRPYFRGVNEMRGRYVGAEISAVDTHPDIPKIPEMSDAICSVWIAKSSTVTATTQSMCGMLTGSALGIITAYSLGWDTTGPRYANGDVTARWVVSPGVPIISLKVDDGFNQKRKSSSRVWAVALNALGEVYYLCDVPVGKPGRTTGEDMTRNAWYAGRTAYWHLLEATRRVAREDEADKNATRGGYSPRSPSLDMHLSKEQIVAEAREIEKFMRYRPSHFRKVCEGWDMQRKLEVDFASDDGKGAGESIFVIDCGLAENRPVSIQRYSRSLIPVQGTPSESSTPLAPVPTSLFGSIGGFANRISPVSESQAPLSPPPTPKSPPVPSTVLHDWFKQGFEFKGHGHDTILSVALDNSLTSLYTLGEDPLHTANEASSTTSPWAEHGAREIPGRRTRFIIAGTNSGAVLVWNARDDDRTRDIQPLRILQTESPEVSAVAASGLYLVHGGSDGLVQAWDPLASTTDPIRTINARSNGRVPRHMLVMNPALQEETYSAAKAIYLDPDSTTLQGVVSFGAFLRYWSYGSNGHATGRKRRVRHADMDARLASRRQGHAVSGYIASEEAEMRREDEQQAREHNRRLKRFGALGDLTEEEALLYAQMVSQEAYHVEEQRRASDSAADASLDTASSFSENTVETLTPDPSVADPVASETSGMAEDDEYEQQIQQAIRLSLLEGVNNGVEQSPVDSSRGNSSVDFDQPVNVKYKPKGGKKGKQSGASSGGSPSASHTPVGGGASSSRLSTTEDEDLAIALSLSMQDQGGGYSPPGMSSSTMMGRSAYLEAAAGVDEEDEFPSLPGEGKGKGVQRW

SEQ ID NO: 7 - protein, 859 aa, Trichoderma reesei
MPHRERGKQREGGDSYRPSRPARSRSRSRSPPRAPVPVRTEEEKQAAAKAEYEKLLNMRSGGTYIPPARLRALQAQITDKSSKEYQRMAWEALKKSINGLINKVNTANIKHIVPELFGENLVRGRGLFCRSIMKAQAASLPFTPIYAAMAAIVNTKLPQVGELLVKRLIMQFRKGFKRNDKAVCLSSTTFLAHLINQQVQHEMLAGQILLLLLHKPTDDSVEIAVGFCKEVGQYLEEMQPAISMAVFDQFRNILHESDIDKRTQYMIEVLFQIRKDKFKDHPAIKEELDLVEEEDQITHKVELDGEIDVQDGLNIFKYDPEWEEHEEAYKRLKAEILGEASDDEEGDEDEDEDESSEDEENEETKAMEIKDQSNADLVNLRRTIYLTIMSSADPEEAVHKLMKINLPVGQEPELPSMIVECCSQEKTYTKFFGLIGERFAKINRLWCDLFEQAFVKYYETIHRYENNKLRNIAMLFGHMFASDALGWHCLSVIHLNEEETTSSSRIFIKILFQHISEEIGLAKLRARMTDETLRPSLEGLFPRENPRNIRFSINYFTSIGMGVLTEEMREHLMNMPKPALPAPAAQDRSDTDSVSSYSSYTHSSYSSRSRSRSRSVGRRSGGRGRSLSRTPPRRGARSRSYSDDSRSPSRSRSRSRSDSVSTRGRRRASYSASPPRRGGRRVASRSRSYSSGSSRSPPPRNRGRARSNSYSSYSRSPSSSPRRGRDADSASPPPRRGRPRQSPPGGPAGRRNSSSVGSGGPRKKPRRDSRSPSRDYSSRSPSRSPSRSRSPPPAARGRRGSYTPSRSRSPPPRRVRDGSPGRLRGGRSPSPPLPVKRRRYDSESVSRSPPPLKRGRRDN

SEQ ID NO: 8 - DNA, 2580 bp, Trichoderma reesei
  source 1..2580: mol_type = unassigned DNA
atgccgcaccgcgagcgcggcaagcagcgagaaggcggcgactcgtaccgcccctcgaggccagcgcgttcacgctcgcgctcgcgatcgccgcctcgcgcgccggtgcccgtgcggacggaggaggagaagcaggcggcggcaaaggccgagtacgagaagctgctcaacatgcggtcgggcggcacgtacatcccgccggcgaggctgagggcgctgcaggcgcagatcacggacaagagcagcaaggagtaccagcggatggcgtgggaggcgctcaagaagagcatcaacggcctgatcaacaaggtcaacacggccaacatcaagcacattgtgcccgagctgtttggcgagaacctggtgcgcggccgcggcctcttctgccgctccatcatgaaggcccaggccgccagtttgcccttcacgcccatctacgccgccatggccgccattgtcaacaccaagctgccgcaggtcggcgagctgctggtcaagcgcctcatcatgcagttccgcaagggcttcaagcgcaacgacaaggccgtctgtctgtcgtcgaccaccttcctcgcccacctcatcaaccagcaggtgcagcacgagatgctggccggccagatcctgctgctgctgctgcacaagccgaccgacgacagcgtcgagattgccgtgggcttctgcaaggaggttggccagtacctcgaggagatgcagcctgccatttccatggccgtcttcgaccagttccgcaacatcctccacgagtccgacattgacaagcgaacgcagtacatgattgaggtgctcttccagatcaggaaggacaagttcaaggatcacccggccatcaaggaggagctggacttggtggaggaggaggaccagatcacgcacaaggtggagcttgatggcgagattgatgtgcaggacggactcaacatcttcaagtacgacccggagtgggaggagcatgaggaggcatacaagaggctcaaggcggagattctgggcgaagccagcgatgacgaggagggcgacgaggacgaggacgaggacgagagctccgaagatgaagaaaacgaagagacaaaggccatggagatcaaggaccagtctaacgccgacttggtcaacctacggaggaccatctacctcaccatcatgtcgagcgccgacccagaggaagcagttcacaagctgatgaagatcaacctgcccgtcggccaggaacccgagctgccctcgatgattgtcgagtgttgctcgcaggagaagacgtacaccaagttctttggcttgatcggcgagcgtttcgccaagatcaatcggctgtggtgcgacctctttgagcaggcctttgtcaagtactacgagacgatccaccgatacgaaaacaacaagctgcggaacattgccatgctgtttggccacatgtttgcttccgacgctctgggctggcactgcctttccgtcattcacctcaacgaggaggagaccacgtcgagcagccgcatcttcatcaagattctgttccagcacatttccgaggaaatcggcctggctaagctccgggcacgcatgactgacgagacgctgcggcccagcctcgaaggcctcttccccagagagaaccctcgcaacatccgattctccatcaactacttcaccagcatcggcatgggtgtactgaccgaggagatgcgagagcacctcatgaacatgcccaagcctgcgctgcccgcccctgctgctcaggaccgctcggatacggactccgtctcgagctattcgtcttacactcactcatcatactcttcccgctcgcgctcacggtcccgatctgtgggtcgtcggagcggcggtcgaggccgatcgctttcccgaactccgcctcgacgtggcgcaaggagccgatcctactctgacgactcacggtcaccgtcgcggtcaagatcacgatcccgctccgattccgtctctactcgtgggcgaaggcgagcgtcgtactcggccagtcctccccggcgtggtggccgtcgggttgccagcagaagccgaagctactcgtcgggctcctcacggtctccgccaccacggaaccgcggtcgcgcacgaagcaactcgtatagttcctacagccgctctccatcttcttcaccacgacgcggcagagacgcagactcggccagcccgcctccgcgaaggggtcgaccgcgccagagcccaccaggcggtcccgcaggtcgaaggaacagctcgtctgtcggcagcggagggccccgcaagaagccccgacgggacagccgatcgccgtctcgcgactattcgtcccggtccccgtctcggtcgccgtcgagatctcgatcgcctccgccggctgcgcgtggccgaaggggctcttatacgccgtcacgcagccgcagcccgcctccgcgcagggtgagggatggctcgccgggtcgtctgaggggtgggaggtcgcctagtcctcctttgccggtgaagaggaggcggtatgatagcgagagtgtttctcggtcgccgcctcctttgaagcgcgggagaagggataactaa

SEQ ID NO: 9 - protein, 543 aa, Trichoderma reesei
MTVLTSPLASYNVANKLYKTTLLNTVCLVAGLSIFFFGYDQGLMGGVNTTRDYAERMGFGHWDEDQNIVVVDKPLLQGGIVAVYYLPGTLCGCLLGGWLGDRYGRIKTIAIACAWSVCAAALQASAMNANWMFCARVLNGVGTGILNAITPVWATETAAHTSRGQFVSIEFTLNILGVVVAYWLEFGTSKYHDNTSSFIWRFPVAFQILPLILLFLIIWIMPESPRWLVKVGREEEARFILGRLRGNEGEDGLKAEAEYNDIVNIHKLEVDTAKQQSYFSMFFGIGSGKLHTGRRVQLVIWLQILQEWIGIAGITIYGPEIFTIAGISAKDRLWVSGINNITYMFATLICVFTIDRIGRRWTLYWGAVGQGICMFVAGGLARATINASGKASQSHIGGAATFFVFLYTAIFGATWLTVPWLYPAEIFPLQVRAKGNAWGVVGWSIGNGWCVLLLPTIFKALNEKTLYIFGAVNALSILVVWALYPESNQRTLEEMDLVFASDSIWAWEAERNFAKLKAENPDLVQGSTNHGVVDIEQVAEPKE

SEQ ID NO: 10 - DNA, 1870 bp, Trichoderma reesei
  source 1..1870: mol_type = unassigned DNA
atgaccgtcctcacctcacctctggccagctataatgtggccaacaagctgtacaaaaccactctgctcaacaccgtctgcctcgtggccggactgtcgatcttcttcttcggctatgatcagggattgatgggcggtgttaacacgacgcgcgactatgccgagcgcatgggctttggccactgggacgaagaccagaacattgtcgtcgtcgataagccgctgctgcagggcggtatcgtagctgtctactatctccccggaacgctgtgcggttgtctgcttggcggttggcttggtgatcgctatggccgtatcaaaacaattgccattgcctgtgcgtggagtgtctgcgcagccgccctgcaggcctcagctatgaatgcgaactggatgttttgcggtatgtcgatgattcttggacaatcacaaccgaactattactgatgatgagatgaaacagcccgcgttctgaacggcgtcggcactggaatcttgaacgcaatcacgcctgtgtgggcaaccgagactgctgctcacacttctcgaggccagttcgtttccattgagttcaccctcaacattcttggtgttgttgtagcctactggctggaattgtacgtgcctcctcactcaggatccccagtcttgtggaaagtctccctaatgcggtggcagtggtacttctaaatatcacgacaacacatcctccttcatctggagattcccggtcgccttccagatcctccccctaatccttctgttcctcatcatctggatcatgcctgaatccccccgctggctcgtcaaagtgggtcgtgaagaagaggctcgcttcatccttggtcgtctccgtggcaatgagggcgaggacggcctcaaggcggaagcagagtacaatgatattgtcaacatccacaagcttgaagtagacaccgccaagcagcagagctacttctccatgttctttggcattgggtctggaaagctacacactggccggcgcgtgcagctggtcatctggctccagatattgcaagagtggatcggtattgcgggaatcaccatttacggccctgagatctttacgattgctggcatcagcgcaaaggacagactctgggttagcgggatcaacaatatcacatacatggtacgtttagccaacacctcctcacctcaaagattccatcacactaacacgggagcagttcgccacactgatctgcgtcttcaccatcgatcgcataggtcgccgttggactctgtactggggagctgtcggccagggcatttgcatgttcgtcgccggtggcctcgctcgcgcaaccatcaatgcctcaggcaaagcaagccagagccacatcggcggcgctgcaacattctttgtgttcctctacactgccattttcggcgctacctggctgacggttccttggttgtatccggccgagattttccctctgcaggttagagccaagggaaatgcctggggtgtcgttggctggtccattggcaacggctggtgtgtaagtgcacttttcattctcctctcccgtctgggctcttctggtctaatcttctctaggtgctcctgcttcctacgatcttcaaggcgctcaacgaaaagacactctacatttttggcgccgtcaacgccctgtccatcctcgtcgtgtgggctctgtaccccgaatcgaatcaacgaactctagaggagatggacctcgtctttgctagcgacagcatctgggcctgggaggctgagcgtaattttgccaagctcaaggctgaaaacccggatcttgttcagggctcaacaaaccacggagttgtagatattgagcaagttgccgagccaaaggagtag

SEQ ID NO: 11 - protein, 735 aa, Trichoderma reesei
MAVNRIRGAFAAPRKGETFELRAGLVSQYAYERKESIQKTIMAMTLGKDVSALFPDVLKNIATSDLDQKKLVYLYLMNYAKTHPDLCILAVNTFVQDSEDPNPLVRALAIRTMGCIRVDKMVDYMEEPLRKTLRDESPYVRKTAAICVAKLFDLNPAMCIENGFIETLQEMIGDPNPMVVANSVQALAEISETAPETRALLVTPPVLKKLLMAMNECTEWGRITILTVLADYAATDVKESEHICERVIPQFQHVNPSVVLAAVKVVFIHMKSINPELVRSYLKKMAPPLVTLVASAPEVQYVALRNIDLLLQAKPDILSKELRVFFCKYNDPPYVKMQKLEIMVRIANEKNYEQLLSELKEYALEVDMDFVRRAIKAIGQVAIKIEEASGKCVQALEDLLATKVNYVVQEVVVVIKDILRKYPGYEGVIPSLCNYIDELDEANARGSLIWIVGEYAEKISNAEEILEGFVDTFLEEFTQTQLQILTAVVKLFLKKPSGAQGLVQKVLQEATTNNDNPDIRDRAYVYWRLLSGDLEVAKNIVLSQKPTISTTMTSLPTALLEQLLSELSTLASVYHKPPEAFVGKGRFGADEIQRAAIQEQRQNAAENPIAASVAAAAANGSSSVSQNNIENLLDIDFDGAAPASQEQNSAAGTPDRVSSPATGGMADMMSMFDAPPAGSSGGAPSGGMNDLMNGFEGLNFGATSTNQPLPAAMQLHNAQGGSQPKKDSDDLLGLL

SEQ ID NO: 12 - DNA, 2589 bp, Trichoderma reesei
  source 1..2589: mol_type = unassigned DNA
atggcggtgaatcgcatccggggcgcctttgccgcgcctcggaagggagagacattcgagctgcgggccggcctggtgtcgcagtatgcctacgagcggaaggagtccatccagaagaccatcatggccatgacgctgggcaaggacgtgtccgccctgttcccagacgtcttgaagaacattgccacgtccgacctggaccagaagaagctggtctacctctacctcatgtatgtggctgcagacaatggccgaccatgatcacacacacggagcgaaggacgagatactgcctgacgtggcgatgcggtgctaacgtggagtgtgaccccaggaactacgcaaagacacacccagacctctgcattctcgccgtcaacacgttcgtgcaagactcggaagacccgaacccgctggtgcgagcgctggccatccgcacaatgggctgcatccgggtggacaagatggtcgactacatggaggagccgctgcggaagacgctgcgggacgagtcgccgtacgtgcgcaagacggccgccatctgcgtggccaagctgttcgacctgaacccggccatgtgcatcgagaacggcttcatcgagacgctgcaggagatgattggcgacccgaaccccatggtggtcgcaaactcggtccaggcgctggccgagattagcgagacggcgcccgagacgcgggcgctgctggtgacgcccccggtgctcaagaagctgcttatggccatgaacgaatgcaccgaatggggtagaatcaccattctgaccgtgctggcagactacgctgccaccgacgtcaaggagtcggagcacatctgcgagagggtcattccgcagttccagcacgtcaaccctagcgtggtcctggctgctgtcaaggtggtctttattcatatgaagtcgattaacccggagctcgtgcggtcatatcttaagaagatggcgcctccactcggtgcgttccgatcatgtccccgatttgacatctgagaagacatgacgtgactatgctaacactgcagcttgtatacagtcacactggttgcttctgcccccgaggtccaatacgtcgctctcagaaacattgatctgctccttcaagccaagcccgacatcctgagcaaagagttaagagtcttcttttgcaaatacaacgacccgccgtacgtcaagatgcaaaagctggaaatcatggtcaggatagcaaacgaaaagaactacgagcagctcctgtctgagctcaaggaatacgccctggaagtggacatggactttgtgcgccgagccatcaaggccatcggccaggtggccatcaagattgaggaggccagtggcaagtgcgtgcaggcgctggaagatcttctcgctaccaaggtcaactacgtggtgcaagaggttgtcgtggtcatcaaagatatcctgcgaaagtaccccggttacgagggcgtgatcccctcgctctgcaactacattgacgagctcgacgaggccaatgctcgtggatccctcatctggattgtgggagagtacgccgagaagattagcaacgctgaggagattctggagggttttgtagacacctttttggaggagttcactcaggtatgtggagagctgtggaaaagtcggggattttggctaatcgaactgcagacacaactccagatccttacagctgttgttaagctgtttttgaagaagccgagtggcgcgcagggcctggttcagaaggtgctgcaggaggcaacaaccaacaacgacaaccccgatatccgcgacagagcatacgtctactggcgattgttatcgggagatttggaggtggccaaggtaggagtcgttggcgtcctttgatgagagctgcgcatactgacggatctcaagaacattgtcctgtcacagaagccgaccatttcaacaacaatgacaagcctgccgactgcgctactggagcagctgctgtcggagctgtcaactctggcgtcggtataccacaagcccccggaagcctttgtcggcaagggccggttcggtgccgacgagatccagcgagccgccatccaggagcagcgccagaacgccgcggaaaaccccatcgccgcatccgtggctgccgccgccgccaatggctcctcgtcggtctcgcaaaacaacattgagaacctgctggacattgactttgacggcgcagcaccggcctctcaggagcagaacagcgcggcgggaacacctgaccgggtgtcgagcccggccacgggtggcatggccgacatgatgagcatgtttgatgcgcctccggctggcagctctggaggtgctccgtccggcggcatgaacgacttgatgaacggatttgaggggctcaactttggggccacgagtacaaatcagccgttgccggcggcgatgcagctgcacaatgcgcaaggcggctctcagccgaagaaggatagcgatgatcttttgggtttgttgtaaatgttggaggagcgtatatgcatgcaagcagcaagccagaaggggagaagaatcgacaagagagactggaggaggaggcaagggaggggggggg

SEQ ID NO: 13 - DNA, 5634 bp, synthetic construct
  misc_feature 1..5634: note = DNA fragment for transformation
  source 1..5634: mol_type = other DNA
cttaagactgaaagtaggtggcaatctgggttgtgcgtccatggcgggcatcaagggctgcatgggcagttgatcttcgcatgggatcaccaaccttcatctctgcttggccatgataggctgcttgtgtgtatcacggaggaatactctgaaccgacgtggtagaacattcaagtccgggctagccccgtgtagccccaaaggcagcttgagctttgaatctgtctggaaccgggaagcatggggggagggaagcaaggaactggcaaggaataggacagaaacgcacagacgcagaaaaaagggacaggaatggaactctcctttgctccgtatctcctgattgagacagatgggggatggggatgagacgccgccgacgtaagactgcgagctaccaaaggcagcttagcacggacgagctggataaagggttcatgtgccattgctttttttgtcatgtgaaagtcgagctgttggtgatgcagctgtcaaagggagagaaggacagagggaggggcactttgacgagaaggctgtcgtgcttttcccctgcaggatcaatcgaacaaacaaacaaacaaagatggtgttcgtcattgttctcattgattctgcttcttccgtccttcactccatcttctattgcctagcctcacgtcctcctcctgtgttaccgtactagaaggcctgtcagcaagccaccaatacaggcctttgttattatggggggacttctcatctcacccctcaggtaagatcgatctaaagtacctggtaatgatgcgcccagcctagtggactgacagcagtactggctttgtctggctgctgaagacgaggcatctggttggtgcttttgtggtgatatctcgaatgttccgcattcagcctctggtcttggccaacgtagctttgctactgctgcaacattcaccttggtgagtctccaattggacggtgagtatcaactggggaagcctcagctgtccacttgaacggtcaattgtatctgcgtagccagtgattggtagatgggcttgcttggattcatcaacctccttcaccagccctgctgctctttgtcaagtctctctcctcctctttgtcgtgctacctacagcagtattacgtacagggcgctccccgtattacctaagtccctatctcggcagcggccgcctagtcatcattggataggcagattactcagcctgaatgacatcaacatgttacccatgatacaataggtcacacaaacaagcgctaagatgcacttggtatgacaagcccagtagtccgtttcaaaagacctagatgatgaactacaacatgaggtgttgcctcctgatccagtccaactgcaaacgctgatgtatactcaatcaagcctgatgtaaatgctgcgactcgattcgctggatatgaagatcaaagagagctctgatgggtccaatatagccgggttttgttaggacagtccaccacaccgatattagaattggtcaagcaccttatcatttcatagagattgcggtttctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcgggcatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacgcgttcttcttcaaccaagagttgtccaaggggatactgtcgcttgtacctccagacaaccctgtacacagagatggtttgtcacacgaaacaatcaggacgatacgctattcatgggagacgatgaaactgacaatgagggcttcaagtcatgacactccagggcgaaatggtgaggcaggcagaaatgctgcgctcgctcgacgagccgcacgagcctgtctattccgccaggcaacagttcttcggcacggtcgacaacgcccccgtgtccccgcggcagcttcctcaagacgacaacaggcgagtcacgctcaacgttccgcagggccggagtcagccgtcgtaccgaccagccgtcccctcgaacctgtccgccggcacgcggcgaccctacggatccatcagcggcggtgccggctcctcacctctgcgcaatgccgcgccggcacccccagcagggccgcatcccctttccaacgtggagaccgtccccagcaacctggccagacgccataccgccgcagacattcgcgcacacggatggcagcccaacgcaccgccttatccccctggagccgtgccgccgcccgtatggcctcaatctcccaatcggcccgaggatcagaggataagagactctctctcttcgttttccctgcaggccccatcggctcatggacacccgcactcgcggccggcgacgccaccccacgctcccttttcaaacggcgcgagcggagggtcggatacctttggcagctggtcgtggggcgcggcatcgaaccgcgagagcaagacgattggagcgttgaaggagtcgtcagcgccgcccaccaggaggggcagcatggcacacatcctcaaccccagcgataccgctgagcgatcagacgaggacgaggacccccggggcgacgacgacagaaagcgaaaacgaagacagtgatgccgcgccgtgtagcgttgaagaaaagagggggactggatcatggccatggcacatgattttggcgctggttacaacaaggagtctctttaatccaggctggcgtttttttcgaaatatccctgctccgcactggttcctggatgtcttcaggtttccatccgttgcgttttcattccttctaccgcatctttgtctcatttcccgacatttcagattggatatcttggcgctgcagtaagtctgtaagctatgctcattgatgacggcctcggagaatacactttcgcctttacgccttgattgtatattagcacatattcctgaaccggatcatggccataggcttggcaccacacgtccctcaacaaatgcggacaaaagttgctgtatcaaggaatcgtcgatactagt

SEQ ID NO: 14 - DNA, 37 bp, synthetic construct
  misc_feature 1..37: note = Oligo DNA for PCR
  source 1..37: mol_type = other DNA
atgcatcttaagactgaaagtaggtggcaatctgggt

SEQ ID NO: 15 - DNA, 35 bp, synthetic construct
  misc_feature 1..35: note = Oligo DNA for PCR
  source 1..35: mol_type = other DNA
atgcggccgctgccgagatagggacttaggtaata

SEQ ID NO: 16 - DNA, 33 bp, synthetic construct
  misc_feature 1..33: note = Oligo DNA for PCR
  source 1..33: mol_type = other DNA
atacgcgttcttcttcaaccaagagttgtccaa

SEQ ID NO: 17 - DNA, 33 bp, synthetic construct
  misc_feature 1..33: note = Oligo DNA for PCR
  source 1..33: mol_type = other DNA
atactagtatcgacgattccttgatacagcaac

SEQ ID NO: 18 - DNA, 5385 bp, synthetic construct
  misc_feature 1..5385: note = DNA fragment for transformation
  source 1..5385: mol_type = other DNA
cttaagatgctgcaccaagttgctatgaatcatggggctgactatgactgatgcatttgtggtatgatatatagacgcaccaccttgtagtgtccagtagtatcatcttttactaccacaatcaatgagatatgaagagacgaagacgcaactcagcagaacagaagcatgccctgctgactcagcaggctgcgggtgacgacaacgatccccctttggctgcgttatggtttcatggctgcatctctccaaaactcagaagagaacaaagagccaggcagccggccagctacctattctacctacctaccttacttgaccacctaatgaaactcaccaacattccaccctctgcaaccattctgtacctcctcaattgatgtgctccttgtgataccccgatttatccttgcaatcctgttgattgcgcatttgattgattgatgaattgattgattgctcctacctttgatacccttcatcacaccatgggccaagccccttctcagacgcggcggactcggcggacgcacgaggaactgacacaggagcttgtgagcgcgccgttgcaccttggcctgggaacaagcaagggcctaggctttgctaactgtgcgtgaacaggcgtacagattcaaggaaaagtgcttcacgtcgctggagtactactcactgaaggatgtcttcaagaaactggccgaccagcagggcgacatacggtatctcaaggaggacaccatagctcgcttcctcgagatcccagacatcctcggggcttcgccggtcatcttccacatgatctcgtatctgggtgcgtttccctttctgcaggaggcccccgtggtgctcgagctggcgcagttgatcatggtcgttgtcatcatgacggagcgatataagcgcgttcttgcgaagggctcgacagacaggacgaaattattttttaaaagcttggctgtgtatgaccggaaagtgatggaggagaccggttcttcaccgcggaattccacttcaaaagacgccactgcgaggccgagcgccaacacaagaggctttgcaatcgacgagccgatggccgaggacgaggatgatggcgacgacgacgacgatgatcttgtcattagcggccgcctagtcatcattggataggcagattactcagcctgaatgacatcaacatgttacccatgatacaataggtcacacaaacaagcgctaagatgcacttggtatgacaagcccagtagtccgtttcaaaagacctagatgatgaactacaacatgaggtgttgcctcctgatccagtccaactgcaaacgctgatgtatactcaatcaagcctgatgtaaatgctgcgactcgattcgctggatatgaagatcaaagagagctctgatgggtccaatatagccgggttttgttaggacagtccaccacaccgatattagaattggtcaagcaccttatcatttcatagagattgcggtttctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcgggcatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacgcgtagagtgtgtactagcatccttcgtcgatgtcgagacgtcgccaggcattggatatacccggttcaagactgtcattccggtcctattccccaacctctttgctgggttcaatggcctgtttgaacactttcttttctccaaggatctggacttctcgaagcacaaggttgagaagcctggtgacgagcagctcatcattggcaagattgcgcaacctctgctcccaacccctggcgacatcatgactgagcatacgctgtcgcaactttcactgtttctacctggctcctctctgttccgaagagtgagattgctctattcgggaaacgatgctggattctccatgggcagcctccagaccaaggtctttaactggagagccccaaccattcttctagtcagcggatcgagattagcagacgtccccgagggaggccaagaggcatcgttcgcctcttcgcttcccaccaaacggttccctcatggtagtaaatccgagcgtgtaacgtttggcgtgtacgttcgagagccttggaagcacacgcacaaagagtgcttcggcaattcggaaacaatactctttcaactagaacccattcacgatgttttccctgcctctacaatcaatacagactacgtcaccttcacgaaaccacctggcaaccggccctgtctagcatttgggtgcccacaccccaaaccgacgcagtcgcatcgcaaggagggcatacatgctttaggagccgtgtctctgttgtttgatgaatctttcgagttcggagtcttcaatcatgactacaagtcgagagggggcgctttccacactagcatcgtgaggaaatatgattttcaggatcggtttcgaatcgagaacatggaagtctggggatgtggtggtgacgaggaggccaaggcgcaagcagagagatgggcttgggaagaacgtgaagcggaagcccgtcgcaggatcaaccttgggacgggtgacattgaggcggacagggcattgctggagatggccgggctagtaggagggaatcgcagcggcggttcaatgggttgaatggatgatcacggaatgatacgatgtaaacttctgtgttctaggtcgcgggcactggattctcccctgatatcatgatatgcttttgggtaactagt

SEQ ID NO: 19 - DNA, 1131 bp, synthetic construct
  misc_feature 1..1131: note = Synthetic gene for transformation
  source 1..1131: mol_type = other DNA
cttaagatgctgcaccaagttgctatgaatcatggggctgactatgactgatgcatttgtggtatgatatatagacgcaccaccttgtagtgtccagtagtatcatcttttactaccacaatcaatgagatatgaagagacgaagacgcaactcagcagaacagaagcatgccctgctgactcagcaggctgcgggtgacgacaacgatccccctttggctgcgttatggtttcatggctgcatctctccaaaactcagaagagaacaaagagccaggcagccggccagctacctattctacctacctaccttacttgaccacctaatgaaactcaccaacattccaccctctgcaaccattctgtacctcctcaattgatgtgctccttgtgataccccgatttatccttgcaatcctgttgattgcgcatttgattgattgatgaattgattgattgctcctacctttgatacccttcatcacaccatgggccaagccccttctcagacgcggcggactcggcggacgcacgaggaactgacacaggagcttgtgagcgcgccgttgcaccttggcctgggaacaagcaagggcctaggctttgctaactgtgcgtgaacaggcgtacagattcaaggaaaagtgcttcacgtcgctggagtactactcactgaaggatgtcttcaagaaactggccgaccagcagggcgacatacggtatctcaaggaggacaccatagctcgcttcctcgagatcccagacatcctcggggcttcgccggtcatcttccacatgatctcgtatctgggtgcgtttccctttctgcaggaggcccccgtggtgctcgagctggcgcagttgatcatggtcgttgtcatcatgacggagcgatataagcgcgttcttgcgaagggctcgacagacaggacgaaattattttttaaaagcttggctgtgtatgaccggaaagtgatggaggagaccggttcttcaccgcggaattccacttcaaaagacgccactgcgaggccgagcgccaacacaagaggctttgcaatcgacgagccgatggccgaggacgaggatgatggcgacgacgacgacgatgatcttgtcattagcggccgc

SEQ ID NO: 20 - DNA, 33 bp, synthetic construct
  misc_feature 1..33: note = Oligo DNA for PCR
  source 1..33: mol_type = other DNA
atacgcgtagagtgtgtactagcatccttcgtc

SEQ ID NO: 21 - DNA, 33 bp, synthetic construct
  misc_feature 1..33: note = Oligo DNA for PCR
  source 1..33: mol_type = other DNA
atactagttacccaaaagcatatcatgatatca

SEQ ID NO: 22 - DNA, 5859 bp, synthetic construct
  misc_feature 1..5859: note = DNA fragment for transformation
  source 1..5859: mol_type = other DNA
cttaagtctgatgatgaacaaatggctcaagggcatgcatgcaacccgggaaatgaccatccctgctctacacgaaagacacacaagaaaaaagggaggcgtcccgtccaactgggttcccgctttgcgctagtaatcaggctttttttttctcgacatgccaacaaagccagtgtttctcaactgtatcgattattagtggaaaattcgccaaggccgaggtgacatgctcccggagcagcgaaaagcagctgggacaaagttgtggcactttgacgccaatagcgggacccgggatacatatgtaggaagcaggcgatgcgggagactgtgtacgagcgacgactgggttccctgacggatagtatcagttgtccgagaagccatgtttaccccattctgctgacggtgtttttgttgggtgggtgttgttcttcagaggacatgcggagagcgcaagtcagcgacgggaccgcggataactccgacaagccttgtcctaggagttcagcgttcttgtttcctgattatagtataaccgcgacagcagaacacctcttgtctctagagtctatcgcggggagggagtctctgagggtaagctgtgggagctgacatctagtctgcctttttagtcccgggctttgagatgcggcattacccgatggtgatgagagatgacttgttctactcaaggaaaaatgacagatgcttagtagcaaaagttggccgaagctaccttgctacctaacctgaaggattttttgccgccttttggcaacttcacacacacggagatccttgtctagctgcgtctctagttcagcttcttggtggactaaatcaagaggaagtcctccactgggcacaataggccgcttggagaaggatagatccgtgaggcctagtctgctgatcggaagatacctgatcacctaagagacgtgagcgttaggatccaagatttgctagatcccatcaactgttaatggcgcgacgatgctactgtttgatgcgcagcggctgagccttggccaatctatggccgtttttgccggaagctggatgccgtcaaaggggtttgctatttgaggcgggtgactgcagaggatgggcacaattgagcagtactcgtacaccacaaaagatataaggtacctagcgaagggtgaagccaggcgggacgaggcactagtgggcaaatggtctgttccggataatctctctgcactgttgggttggttcgtctcacaggcatttgacttgatggaggctcctgagctattgatcgtgctcctagcatggtcgtgacaggagcagcagggacggatgaaggctcattggctgacagtgagttggtgaactgtgattatgacgatgttttgttttcggcagagcatggatggtaagtgcttatctcggcggccgcctagtcatcattggataggcagattactcagcctgaatgacatcaacatgttacccatgatacaataggtcacacaaacaagcgctaagatgcacttggtatgacaagcccagtagtccgtttcaaaagacctagatgatgaactacaacatgaggtgttgcctcctgatccagtccaactgcaaacgctgatgtatactcaatcaagcctgatgtaaatgctgcgactcgattcgctggatatgaagatcaaagagagctctgatgggtccaatatagccgggttttgttaggacagtccaccacaccgatattagaattggtcaagcaccttatcatttcatagagattgcggtttctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcgggcatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacgcgtcatttctcctgtttctgaatcccaagcgccattgtctccgccacctactcctaaatcgccccctgtgccttcgacggttcttcatgactggttcaaacaagggttcgagttcaagggccacggtcacgataccatattgtcagttgctttggacaattcgctcacctcgctctacacacttggggaggaccctctacacaccgcgaacgaagcgtcctccacgacgagcccctgggctgaacacggggcgagggagattcctgggcgccgaacccgcttcatcatcgctggaaccaactccggcgcggtgcttgtctggaatgcgcgtgatgacgatcgaactcgtgatatacaaccactgcgcatccttcagaccgagtcaccagaggtttctgctgtggcagcctctgggttgtatctcgttcacggcggcagcgacggccttgtccaagcctgggatcccttggcatctacaacggatcccatcagaacgattaacgctcggtcaaatggccgggtcccccgtcacatgctggtaatgaatcccgcattgcaggaggagacatactcggcagcaaaggccatatatcttgaccctgattctacgacgcttcaaggtgtagtctcttttggcgcattcctgcgatattggtcgtatgggtccaatggtcatgccacaggtcgcaagcggcgcgtccgacacgccgatatggacgctcggcttgcgagtcgccggcaaggccatgcagtgtcaggctacattgcctctgaggaagccgagatgcgacgggaggatgagcagcaggctcgcgagcacaaccgtcgtctcaagagatttggcgctctaggcgacttgaccgaggaagaagcgcttctctacgcccagatggtctcccaagaggcgtaccacgtagaggagcagcgacgggccagcgattcggcagccgacgccagcctggacaccgcctcttcctttagcgagaataccgtcgagactctgacacctgatccgagcgtcgccgatccggtcgcttcggaaacgagcggcatggccgaggatgacgagtacgagcagcagattcagcaggctatacgtctgtctctgctagaaggcgtcaacaacggcgtggaacagtcacctgtggattcctcacggggcaacagctctgttgatttcgaccaaccggtcaatgtcaagtacaagcccaagggcgggaagaaggggaagcaatcaggggcttcttctggtggctcaccgtctgcgagccacacgcccgttggtggtggtgcttcttcttcgcggctgagcacgactgaagatgaggatttagctcgag

SEQ ID NO: 23 - DNA, 37 bp, synthetic construct
  misc_feature 1..37: note = Oligo DNA for PCR
  source 1..37: mol_type = other DNA
atgcatcttaagtctgatgatgaacaaatggctcaag

SEQ ID NO: 24 - DNA, 35 bp, synthetic construct
  misc_feature 1..35: note = Oligo DNA for PCR
  source 1..35: mol_type = other DNA
atgcggccgccgagataagcacttaccatccatgc

SEQ ID NO: 25 - DNA, 33 bp, synthetic construct
  misc_feature 1..33: note = Oligo DNA for PCR
  source 1..33: mol_type = other DNA
atacgcgtcatttctcctgtttctgaatcccaa

SEQ ID NO: 26 - DNA, 32 bp, synthetic construct
  misc_feature 1..32: note = Oligo DNA for PCR
  source 1..32: mol_type = other DNA
actcgagctaaatcctcatcttcagtcgtgct

SEQ ID NO: 27 - DNA, 46 bp, Trichoderma reesei
  source 1..46: mol_type = unassigned DNA
gcgcacgtgtgccggtcgcgcggctacaagctggtcatctacatgc